(19) United States
(12) Patent Application Publication
(10) Pub. No.: US 2019/0334092 A1
(43) Pub. Date: Oct. 31, 2019

(54) DISPIRO-OXEPINE/THIAPINE DERIVATIVES FOR OPTOELECTRONIC SEMICONDUCTORS

(71) Applicants: QATAR FOUNDATION FOR EDUCATION, SCIENCE AND COMMUNITY DEVELOPMENT, DOHA (QA); ECOLE POLYTECHNIQUE FEDERALE DE LAUSANNE, LAUSANNE (CH)

(72) Inventors: KASPARAS RAKSTYS, LAUSANNE (CH); SANGHYUN PAEK, LAUSANNE (CH); MUHAMMAD SOHAIL, DOHA (QA); KLAUS H. DAMEN, DOHA (QA); MOHAMMAD KHAJA NAZEERUDDIN, DOHA (QA)

(73) Assignees: QATAR FOUNDATION FOR EDUCATION, SCIENCE AND COMMUNITY DEVELOPMENT, DOHA (QA); ECOLE POLYTECHNIQUE FEDERALE DE LAUSANNE, LAUSANNE (CH)

(21) Appl. No.: 16/315,548

(22) PCT Filed: Jul. 7, 2017
(86) PCT No.: PCT/US2017/041060

§ 371 Date: Jan. 4, 2019

Related U.S. Application Data
(60) Provisional application No. 62/359,658, filed on Jul. 7, 2016.

(51) Int. Cl. H01L 51/00; H01L 51/42

(52) U.S. Cl. CPC H01L 51/0061; H01L 51/0073; H01L 51/4226; H01L 51/0068; H01L 51/0074

(57) ABSTRACT

The dispiro-oxepine/dispiro-thiapine derivatives for optoelectronic semiconductors is a compound based on a structure having a functionalized dispiro compound of formula (Ia) or (Ib) with the core unit being a seven-membered heterocycle oxepine or thiapine, the derivative being formed by combining (Ia) or (Ib): with two moities selected from K1 and K2: The derivative is used as a hole transporting material in an optoelectronic and/or photoelectrochemical device.

### TECHNICAL FIELD

The disclosure of the present patent application relates to optoelectronic semiconductors, and particularly to dispiro-oxepine/dispiro-thiapine derivatives for optoelectronic semiconductors that serve as an efficient hole transporting material when applied as a coating on an electron transporting material infiltrated with a perovskite absorbing material to form semiconductors for perovskite solar cells and other optoelectronic devices

### BACKGROUND ART

The conversion of solar energy to electrical current using thin film third generation photovoltaics (PV) has been widely explored for the last two decades. The sandwich/monolithic-type PV devices, consisting of a mesoporous photoanode with an organic/inorganic light harvester, redox electrolyte/solid-state hole conductor, and counter electrode, have attracted significant interest due to the ease of their fabrication, their flexibility in the selection of materials, and their low cost effective production.

In recent years, perovskite-based solar cells (PSCs) have become the hottest topic in the photovoltaics field, since they have inexpensive precursors, simple fabrication methods, and remarkably high power conversion efficiency (PCE) values. A typical PSC configuration is composed of an electron transporting material (ETM), which is infiltrated with the perovskite absorbing material and coated with a hole transporting material (HTM), which plays an important role to facilitate the movement of holes from perovskite to the gold as a back contact.

Recently, bulk layers of organometallic halide perovskite based on tin (CsSnX₃), or lead (CH₃NH₃PbX₃; X=Cl, Br, I) have been introduced as semiconducting pigment for light harvesting, resulting in high PCEs. These perovskite materials show exceptional characteristics, including large panchromatic absorption and very good charge-carrier mobility values as compared to amorphous silicon. Minimizing energy losses while favoring charge-extraction rates is fundamental to take advantage of the intrinsic properties of the perovskites and to improve their efficiency.

Therefore, perovskite-based and other types of solid state solar cells generally contain an organic HTM layer for transporting holes created by charge separation at the light harvester to the counter electrode and/or cathode for filling up with incoming electrons, thereby closing the electric circuit and rendering the devices regenerative.

Spiro-based organic semiconductors have attracted considerable attention, more precisely, 2,2′,7,7′-tetrakis-(N,N′-di-4-methoxyphenylamine)-9,9′-spirobifluorene (spiro-OMeTAD) has been selected as the benchmark HTM for PSC. Currently, most performing solid-state devices use doped spiro-OMeTAD as a HTM. The relatively low PCE of solid-state devices was often ascribed to the low hole mobility in spiro-OMeTAD, which causes interfacial recombination losses by two orders of magnitude higher than in electrolyte-based, dye-sensitized solar cells (DSCCs).

Further, the use of spiro-OMeTAD as a hole transporting material may trigger instability in such solid-state solar cells. Because spiro-OMeTAD has two oxidation potentials that are close, this HTM in the oxidized form is able to form a di-cation, which, in turn, can dismutate and might cause device instability. Further, since spiro-OMeTAD is present in a semi-crystalline form, there is the risk that it will (re)crystallize in the processed form in the solar cell. In addition, solubility in customary process solvents is relatively low, which leads to a correspondingly low degree of pore filling. Along with stability issues, the high cost due to a complicated synthetic route and the high purity that is required (sublimation grade) in order to have good performance have been the main drawbacks for commercial applications of solid-state solar cells.

Due to the tedious multi-step synthesis of spiro-OMeTAD, which makes it prohibitively expensive and cost-ineffective, as well as the necessary high-purity sublimation-grade spiro-OMeTAD required to obtain high-performance devices, there is a huge interest in development of novel small-molecule organic semiconductors.

As such, there have been attempts to find an alternate organic HTM having higher charge carrier mobility and matching HOMO (highest occupied molecular orbital) level to replace spiro-OMeTAD. In most cases, it is difficult to compete with the performance of spiro-OMeTAD-based devices, due to spiro-OMeTAD's unique properties of sufficient hole mobility, thermal and UV stability, and well-matched HOMO energy level to the semiconductor light absorbers. So far, although a large number and different types of HTMs were reported reaching efficiency of 16-19%, only very few candidates have showed PCE values over 19%, mainly because of the additional interaction associated with improving the hole transfer at the HTM/perovskite interface.

Thus, a dispiro-oxepine/dispiro-thiamine derivatives for optoelectronic semiconductors solving the aforementioned problems are desired.

### DISCLOSURE OF INVENTION

The dispiro-oxepine/dispiro-thiapine derivatives for optoelectronic semiconductors are compounds of formula (Ia) or (Ib):

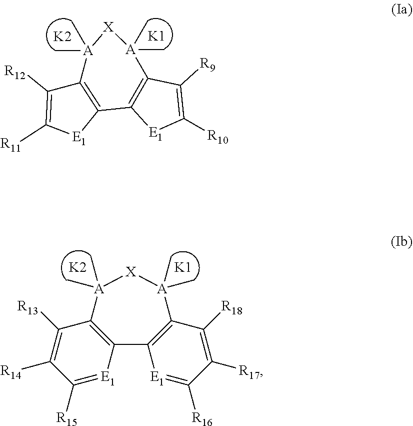

wherein K1 and K2 are the following:

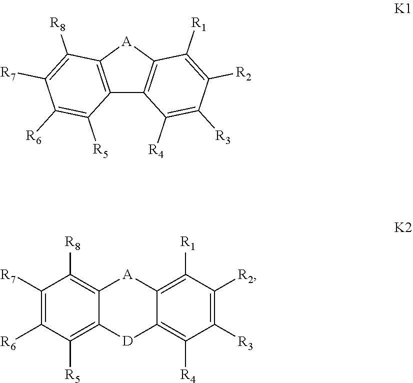

and wherein A is independently selected from Si or C; D is a heteroatom independently selected from O, S, and N and can be bare or substituted by a H, alkyl, or aryl chain to form D-H, D-Alkyl, or D-Aryl, respectively; E₁ and E₂ are independently selected from O, S, and N; X is independently selected from O, S, and Se; at least one R₁, R₂, R₃, R₄, R₅, R₆, R₇, and R₈ is independently selected from substituents comprising 1-50 carbons, 1-20 heteroatoms selected from O, S, N, and 0-2 P-hydrocarbyl, the substituents being further substituted by further substituents selected from H, halogen, C1-C10 alkyl, C1-C10 alkoxy group, C1-C10 alkylthio (—S-alkyl) and —C≡N; wherein any one of R₁, R₂, R₃, R₄, R₅, R₆, R₇, and R₈ different from said substituents comprising 1-50 carbons, 1-20 heteroatoms and 0-2 P-hydrocarbyl is H; and R₉, R₁₀, R₁₁, R₁₂, R₁₃, R₁₄, R₁₅, R₁₆, R₁₇, and R₁₈ are independently selected from H; halogen selected from Cl, F, Br, or I; C1-C30 alkyl; C1-C30 heteroalkyl; C4-C20 aryl; C4-C20 heteroaryl; C4-C30 alkylaryl group; C4-C30 aryloxy group; or C4-C20 heteroaryloxy group; wherein the heteroatom is independently selected from O, S, N, —P(═O)—, and —C≡N; wherein if the alkyl, heteroalkyl, or alkylaryl comprises 3 or more carbons, the alkyl, heteroalkyl, or alkylaryl may be linear, branched, or cyclic; and from a substituent as defined above for R₁, R₂, R₃, R₄, R₅, R₆, R₇, or R₈ of the substituent R₉, R₁₀, R₁₁, R₁₂, R₁₃, R₁₄, R₁₅, R₁₆, R₁₇, and R₁₈ in K1 and/or K2.

An exemplary dispiro-oxepine derivative for optoelectronic semiconductors of the above formula having the following structure is evaluated more fully below for illustration of the properties of the derivatives:

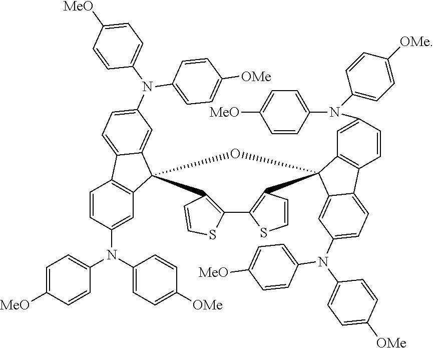

The optoelectronic devices include an electron transporting material (ETM) infiltrated according to the present subject matter disposed on the ETM.

These and other features of the present disclosure will become readily apparent upon further review of the following specification and drawings.

### BRIEF DESCRIPTION OF THE DRAWINGS

FIG. 1A is a general reaction scheme showing two strategies (A and B) for the synthesis of an exemplary dispiro-oxepine derivative for optoelectronic semiconductors referred to herein as DS-HT-SO2.

FIG. 1B is a detailed reaction scheme of strategy A, wherein “a” is Pd₂(dba)₃, ^{t}Bu₃P, NaO^{t}Bu, toluene, 110° C., 94%; “b” is BuLi, Et₂O, −78° C.; “c” is THF, −78° C.->rt, 75%; “d” is FeCl₃, CHCl₃, reflux; and “e” is TFA, CHCl₃, rt, 66%.

FIG. 1C is a detailed reaction scheme of strategy B, wherein “a” is nBuLi, Et₂O, −78° C.; “b” is 1, THF, −78° C.->rt, 80%; “c” is FeCl₃, CHCl₃, reflux, 80% or AcOH, HCl, reflux, 79%; and “d” is Pd₂(dba)₃, ^{t}Bu₃P, NaO^{t}Bu, toluene, 110° C., 81%.

FIG. 1D is a reaction scheme of synthesis of dispiro-oxepine derivatives from intermediate compound 7 of FIG. 1C, wherein a is Ar₂NH, Pd₂(dba)₃, ^{t}Bu₃P, NaO^{t}Bu, toluene, 110° C.

FIG. 1E is a structural formula of dispiro-oxepine derivatives for optoelectronic semiconductors referred to herein as DS-HT-SO2, DS-HT-SO6, DS-HT-SO7, DS-HT-SO8, DS-HT-SO9, and DS-HT-SO10.

FIGS. 2A, 2B, 2C, 2D, and 2E are structural formulas of dispiro-oxepine derivatives for optoelectronic semiconductors referred to herein as DS-HT-SO1, DS-HT-SO2, DS-HT-SO3, DS-HT-SO4, and DS-HT-SO5, respectively.

FIGS. 3A, 3C, 3E, and 3F are plots of Current-Voltage curves of solid state solar cells made with DS-HT-SO2 upon exposure to 100% sun.

FIGS. 3B and 3D are plots of Current-Voltage curves of a solid state solar cell made with DS-HT-SO2, showing a comparison between exposure to 59.5% sun and exposure to 100% sun.

FIG. 4 is a plot of J-V curves comparing the best performing device prepared with DS-HT-SO2 (New HTM) with spiro-OMeTAD (Spiro) as a reference, the devices being masked with a black metal aperture of 0.16 cm² to define the active area, and curves being recorded scanning at 0.01 V s⁻¹.

FIG. 5 is a plot of Current (J)-voltage (V) curves of the solar cell with DDOF collected under AM1.5 simulated sunlight, and were recorded scanning at 0.01 V s-1 from forward bias (FB) to short circuit condition (SC) and the other way round.

FIG. 6 is a set of plots comparing various characteristics of a solar cell prepared using DDOF with a solar cell prepared using spiro-OMeTAD.

FIG. 7 is a reaction scheme for the synthesis route of the DDOF HTM (hole transport material) where the reaction conditions include: (a) LiN(Pr-i)₂, TMSCl, THF, −78° C. to RT; (b) n-BuLi, 2,7-dibromofluorenone, THF, −78° C. to RT; CH₃COOH/HCl, 110° C.; and (c) 4,4′-dimethoxydiphenylamine, t-BuONa, Pd₂dba₃, XPhos, toluene, 110° C.

FIG. 8A are UV-VIS absorption spectra comparing DDOF and spiro-OMeTAD (both absorption intensity [AI] and photoluminescence intensity [PL]).

FIG. 8B are cyclic voltammogram plots comparing DDOF and spiro-OMeTAD.

FIG. 9 is a cross-sectional SEM micrograph of a perovskite device containing DDOF as HTM (hole transport material).

FIG. 10A is a plot of J-V curves comparing the best performing device prepared using DDOF and a device prepared with spiro-OMeTAD.

FIG. 10B is a chart showing the statistical PCE distribution of 30 devices prepared using DDOF as the HTM, the devices being masked with a black metal aperture of 0.16 cm² to define the active area and the curves being recorded with scanning at 0.01 V s⁻¹, a distribution curve fitted by Gaussian function also being shown.

FIG. 11 is a set of plots comparing V_{OC}, J_{SC}, FF and PCE of devices with spiro-OMeTAD and DDOF as HTMs versus time, measured in air at a relative humidity of 10% without any encapsulation for 1000 h.

FIG. 12A is the ¹H NMR spectrum of DDOF.

FIG. 12B is the ¹³C NMR spectrum of DDOF.

FIG. 13A is the MALDI-TOF-MS spectra in a wide mass range of DDOF.

FIG. 13B is the MALDI-TOF-MS spectra in a narrow mass range of DDOF.

FIG. 14A is a thermogravimetric analysis (TGA) plot comparing DDOF and spiro-OMeTAD, taken at a heating rate of 10° C. min⁻¹ in N₂ atmosphere.

FIG. 14B is a DSC second heating curve of DDOF, taken at a scan rate 10° C. min⁻¹ in Ar atmosphere.

FIG. 15 is a graph showing conductivity measurements on OFET substrates of the HTMs (holes transport materials), i.e., DDOF and spiro-OMeTAD.

FIG. 16 is a plot of current (J)-voltage (V) curves of the solar cell with DDOF collected under AM1.5 simulated sun light, the curves being recorded scanning at 0.01 V s⁻¹ from forward bias (FB) to short circuit condition (SC) and the other way round.

FIG. 17 is an IPCE spectra comparison of DDOF and spiro-OMeTAD as a function of the wavelength of monochromatic light.

Similar reference characters denote corresponding features consistently throughout the attached drawings.

### BEST MODE(S) FOR CARRYING OUT THE INVENTION

The dispiro-oxepine/dispiro-thiapine derivatives for optoelectronic semiconductors provide compounds that serve as an efficient hole transporting material when applied as a coating on an electron transporting material infiltrated with a perovskite absorbing material to form semiconductors for perovskite solar cells and other optoelectronic devices.

By “hole transport material”, “hole transporting material”, “charge transporting material”, “organic hole transport material”, “inorganic hole transport material”, and the like, is meant any material or composition wherein charges are transported by electron or hole movement (electronic motion) across the material or composition. The “hole transport material” is thus an electrically conductive material. Such hole transport materials, etc., are different from electrolytes, as charges are transported by diffusion of molecules in electrolytes.

The term “perovskite”, as used herein, refers to the “perovskite structure” and not specifically to the perovskite material, CaTiO₃. As used herein, “perovskite” encompasses and preferably relates to any material that has the same type of crystal structure as calcium titanium oxide and other materials in which the bivalent cation is replaced by two separate monovalent cations.

The dispiro-oxepine/dispiro-thiapine derivatives for optoelectronic semiconductors provide a hole transporting material comprising at least one compound selected from a compound according to any one of formulae (Ia), (Ib), (IIa) (IIb), (IIIa), and (IIIb).

The dispiro-oxepine/dispiro-thiapine derivatives for optoelectronic semiconductors may be used to make an optoelectronic and/or photoelectrochemical device comprising at least one compound selected from a compound according to any one of formulae (Ia), (Ib), (IIa), (IIb), (IIIa), and (IIIb). The optoelectronic and/or photoelectrochemical device may be an organic photovoltaic device, a photovoltaic solid state device, a p-n heterojunction, an organic solar cell, a dye sensitized solar cell, a solid state solar cell, a phototransistor, or an OLED. The optoelectronic and/or photoelectrochemical device may be a solid-state solar cell comprising an organic-inorganic perovskite as sensitizer under the form of a layer.

The dispiro-oxepine/dispiro-thiapine derivatives for optoelectronic semiconductors provide use of a compound of the above formula as a tuner of a HOMO level based on the presence of thiophene groups.

It was found that a compound based on a structure comprising functionalized dispiro-oxepine or dispiro-thiapine structure, highlighted in formula (Ia) or (Ib), is an excellent, highly efficient hole transporting material.

The seven-membered heterocycle is shown below:

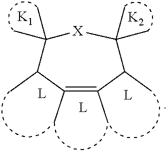

The seven-membered heterocycle comprises one hetero atom X, selected from O or S. L is fluorene or fused aromatics rings with at least one heteroatom, which are connected through the heteroatom X to two fused aromatics rings (K1 and/or K2) having at least one heteroatom. The resulting dispiro-oxepine or dispiro-thiapine structures contribute to both effective charge extraction (HTM function) and photocurrent enhancement (passivation of the perovskite layer, good electron transmission performance and cavity transmission performance) in a solid-state photovoltaic device and improve the PCE of optoelectronic and/or photoelectrochemical devices, particularly optoelectronic and/or photoelectrochemical devices comprising perovskite pigment as a sensitizer. They are non-planar efficient conjugate structures.

The compounds are soluble in organic solvents, despite having a large size, and the solubility greatly facilitates purification, processing, and application or deposition on the sensitizer layer in the solid-state photovoltaic device.

The dispiro-oxepine/dispiro-thiapine derivatives for optoelectronic semiconductors provide a compound comprising moieties of formula (Ia) or (Ib) having two moieties of K1 and/or K2 based on a structure comprising a bi-spiro diarylamino functionalized fluorene and fused aromatics rings with at least one heteroatom. The compounds are selected from a compound with a core of dispiro-oxepine or dispiro-thiapine, of formula (Ia) or (Ib).

The dispiro-oxepine/dispiro-thiapine derivatives for optoelectronic semiconductors may be, for example, a compound of formula (Ia) and may have a different K1 and K2 moiety. The dispiro-oxepine/dispiro-thiapine derivatives for optoelectronic semiconductors may be, for example, a compound of a compound of formula (Ib) and may have a different K1 and K2 moiety. Both formula (Ia) and (Ib) may contain two moieties, wherein K1 or K2 may be the same, or may consist of one K1 and one K2. In general, the two moieties are preferably the same.

As such, the dispiro-oxepine/dispiro-thiapine derivatives for optoelectronic semiconductors is directed to a dispiro-oxepine derivative for optoelectronic semiconductors comprising a compound of formula (Ia) or (Ib):

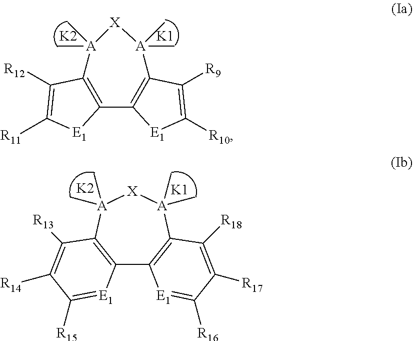

wherein K1 and K2 are independently selected from:

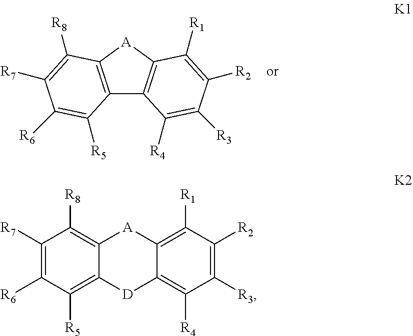

and wherein A is independently selected from Si or C; D is a heteroatom independently selected from O, S, and N, and can be bare or substituted by a H, alkyl, or aryl chain to form D-H, D-Alkyl, or D-Aryl, respectively; E1 and E2 are independently selected from O, S, and N; X is independently selected from O, S, and Se; at least one of R₁, R₂, R₃, R₄, R₅, R₆, R₇, and R₈ is independently selected from substituents comprising 1-50 carbons, 1-20 heteroatoms selected from O, S, N, and 0-2 P-hydrocarbyl, said substituents being further substituted by substituents selected from H, halogen, C1-C10 alkyl, C1-C10 alkoxy group, C1-C10 alkylthio (—S-alkyl) and —C≡N; wherein any one of R₁, R₂, R₃, R₄, R₅, R₆, R₇, or R₈ is different from said substituents comprising 1-50 carbons, 1-20 heteroatoms and 0-2 P-hydrocarbyl is H; and R₉, R₁₀, R₁₁, R₁₂, R₁₃, R₁₄, R₁₅, R₁₆, R₁₇, and R₁₈ are independently selected from H; substituents comprising 1-50 carbons, 1-20 heteroatoms being selected from O, S, N, and 0-2 P-hydrocarbyl; from halogen, which may be Cl, F, Br, or I; from C1-C30 alkyl; C1-C30 heteroalkyl; C4-C20 aryl group; C4-C20 heteroaryl group; C4-C30 alkylaryl group; C4-C30 aryloxy group; C4-C20 heteroaryloxy group, wherein the heteroatom is selected from O, S, Se, N; and —P(═O)— or —C≡N; and wherein if the alkyl, heteroalkyl, or alkylaryl comprises 3 or more carbons, the alkyl, heteroalkyl, or alkylaryl may be linear, branched, or cyclic.

Preferably, A is a carbon atom and is an integral part of the conjugated system or the system of fused aromatic rings. Preferably, E₁ is selected from O and S; E₂ is N; and D is selected from S and N. Any one of R₁, R₂, R₃, R₄, R₅, R₆, R₇, or R₈ may be different from said substituents comprising 1-50 carbons, 1-20 heteroatoms, and 0-2 P-hydrocarbyl is H.

K1 may be a conjugated system and K2 may be a system of fused aromatic rings comprising at least one heteroatom selected from O, S, and N, wherein said aromatic rings may be further substituted by substituents independently selected from H, substituents comprising 1-50 carbons, 1-20 heteroatoms selected from O, S, N, and 0-2 P-hydrocarbyl; from halogen, which may be Cl, F, or Br; from C1-C30 alkyl; C1-C30 heteroalkyl; C4-C20 aryl group; C4-C20 heteroaryl group; C4-C30 alkylaryl group; C4-C30 aryloxy group; C4-C20 heteroaryloxy group; wherein the heteroatom is selected from O, S, Se, N, —P(═O)— or —C≡N, and wherein if the alkyl, heteroalkyl, or alkylaryl comprises 3 or more carbons, the alkyl, heteroalkyl, or alkylaryl may be linear, branched or cyclic.

K1 may be a conjugated system and K2 may be a system of fused aromatics rings comprising at least one heteroatom D selected from O, S, and N, preferably from N and S; wherein said aromatic rings may be substituted by H; halogen selected from Cl, F, and Br; by C1-C30 alkyl or C1-C30 heteroalkyl; wherein the heteroatom is selected from O, S, Se, N; —P(═O)— or —C≡N; by C4-C20 aryl; C4-C20 heteroaryl group; C4-C30 alkylaryl group; C4-C30 aryloxy group; C4-C20 heteroaryloxy group; wherein if the alkyl, heteroalkyl, or alkylaryl comprises 3 or more carbons, the alkyl, heteroalkyl, or alkylaryl may be linear, branched or cyclic; and by 0, 1, or 2 substituents selected from an amino group, P-hydrocarbyl, or a mono- or polycyclic system comprising fused aromatic rings or monocyclic aromatic rings bound together by covalent bond, said ring comprising 0, 1, or 2 heteroatoms selected from O, S, and N; and wherein said amino group, said P-hydrocarbyl and said mono- or polycyclic group may further substituted by H, halogen, R₁, —NR₁R₂, —O—R₁, —P(═O)R₁R₂, or —S—R₁, wherein R₁ and R₂ are independently selected from C4-C20 aryl, C4-C20 heteroaryl, C4-C20 aryloxy group, C4-C20 heteroaryloxy group, C4-C20 alkoxyaryl, C4-C20 alkoxyheteroaryl, C4-C20 aryl aryloxy group, C4-C20 heteroaryl aryloxy group, C1-C20 alkyl, C1-C20 alkoxy group, C1-C20 alkoxyalkyl, C1-C20 alkylthio, C2-C20 alkenyl, and C2-C20 alkynyl; wherein if said alkyl, alkoxy, alkoxyalkyl, alkenyl, and alkynyl comprises 3 or more carbons, said alkyl, alkoxy, alkoxyalkyl, alkenyl, and alkynyl may be linear, branched, or cyclic.

The dispiro compound may be composed from the moieties of formula (Ia) or (Ib) having the two moieties selected from K1 and K2, and may be a compound of formula (IIa), (IIb), (IIIa), or (IIIb),

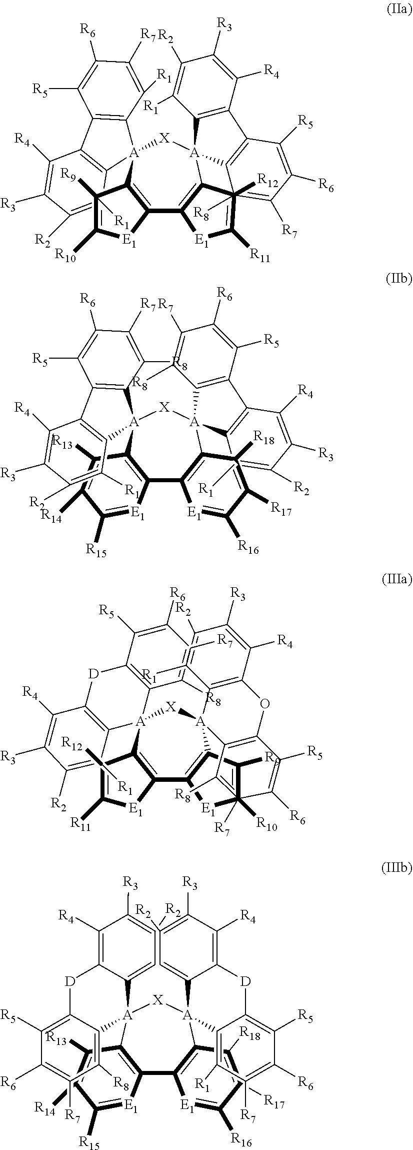

wherein E₁ and E₂ are independently selected from O, S, and N; E₁ is preferably selected from O and S, and E₂ is preferably selected from N; R₁, R₂, R₃, R₄, R₅, R₆, R₇, and R₈ are independently selected from substituents comprising 1-50 carbons, 1-20 heteroatoms selected from O, S, N, and 0-2 P-hydrocarbyl, said substituents being further substituted by substituents selected from H, halogen, C1-C10 alkyl, C1-C10 alkoxy group, C1-C10 alkylthio (—S-alkyl) and —C≡N; wherein any one of R₁, R₂, R₃, R₄, R₅, R₆, R₇, or R₈ may be different from said substituents comprising 1-50 carbons, 1-20 heteroatoms and 0-2 P-hydrocarbyl is H; and R₉, R₁₀, R₁₁, R₁₂, R₁₃, R₁₄, R₁₅, R₁₆, R₁₇, and R₁₈ are independently selected from H; halogen selected from Cl, F, Br, or I; C1-C30 alkyl; C1-C30 heteroalkyl; C4-C20 aryl; C4-C20 heteroaryl; C4-C30 alkylaryl group; C4-C30 aryloxy group; C4-C20 heteroaryloxy group; wherein the heteroatom is selected from O, S, N and —P(═O)—, —C≡N, preferably from O, S, and N, more preferably from O and S; wherein if the alkyl, heteroalkyl, or alkylaryl comprises 3 or more carbons, the alkyl, heteroalkyl, or alkylaryl may be linear, branched or cyclic, and from a substituent as defined above for R₁, R₂, R₃, R₄, R₅, R₆, R₇, or R₈.

All E₁ moieties in the compound of formula (IIa) and (IIb) may be different or identical, preferably identical. All E₂ moieties in the compound of formula (IIIa) and (IIIb) may be different or identical, preferably identical.

The R₉, R₁₀, R₁₁, R₁₂, R₁₃, R₁₄, R₁₅, R₁₆, R₁₇, and R₁₈ substituents of the compounds of formula (Ia) and/or formula (Ib) may be substituents independently selected from H, halogen, cyano group, C1-C20 cyanoalkyl group, C1-C20 alkyl, C1-C20 alkoxy group, C1-C20 alkoxyalkyl, C1-C20 haloalkyl group, C1-C20 haloalkoxyalkyl, wherein said cyanoalkyl, alkyl, alkoxy, alkoxyalkyl, haloalkyl, haloalkoxyalkyl, if they comprise 3 or more carbons, may be linear, branched, or cyclic, and wherein halogen is selected from Cl, F, Br, or I.

Preferably the cyanoalkyl, alkyl, alkoxy, alkoxyalkyl, haloalkyl, haloalkoxyalkyl substituents of R₉, R₁₀, R₁₁, R₁₂, R₁₃, R₁₄, R₁₅, R₁₆, R₁₇, and R₁₈ of the compounds of formula (Ia) and/or formula (Ib) are selected from hydrocarbon, hydrocarbyl, heterocarbon, or heterocarbyl containing from 1-16 carbons, 1-12 carbons, 1-9 carbons, 1-6 carbons, and may contain 0-10 heteroatom and 0-1 halogen selected from Cl, F, Br, or I, and, if they comprise 3 or more carbons, they may be linear, branched or cyclic, preferably linear or branched.

Each R₉, R₁₀, R₁₁, R₁₂, R₁₃, R₁₄, R₁₅, R₁₆, R₁₇, and R₁₈ may be independently selected from a substituent as defined for R₁, R₂, R₃, R₄, R₅, R₆, R₇, or R₈.

At least one of R₉, R₁₀, R₁₁, R₁₂, R₁₃, R₁₄, R₁₅, R₁₆, R₁₇, and R₁₈ may be selected by 0, 1, or 2 substituents selected from an amino group, P-hydrocarbyl or a mono- or polycyclic system comprising fused aromatic rings or monocyclic aromatic rings bound together by covalent bond, said ring comprising 0, 1, or 2 heteroatoms selected from O, S, and N, and wherein said amino group, said P-hydrocarbyl and said mono- or polycyclic group may further substituted by H, halogen, R₁, —NR₁R₂, —O—R₁, —P(═O)R₁R₂, or —S—R₁, wherein R₁ and R₂ are independently selected from C4-C20 aryl, C4-C20 heteroaryl, C4-C20 aryloxy group, C4-C20 heteroaryloxy group, C4-C20 alkoxyaryl, C4-C20 alkoxyheteroaryl, C4-C20 aryl aryloxy group, C4-C20 heteroaryl aryloxy group, C1-C20 alkyl, C1-C20 alkoxy group, C1-C20 alkoxyalkyl, C1-C20 alkylthio, C2-C20 alkenyl and C2-C20 alkynyl, and wherein said alkyl, alkoxy, alkoxyalkyl, alkenyl and alkynyl, if they comprise 3 or more carbons, may be linear, branched or cyclic.

An exemplary dispiro-oxepine/dispiro-thiapine derivative for optoelectronic semiconductors is 2,2″,7,7″-tetraalkyldispiro[fluorene-9,4′-dithieno[3,2-c:2′,3′-e]oxepine-6′,9″-fluorene], having the formula:

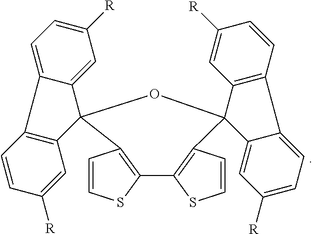

Another exemplary dispiro-oxepine/dispiro-thiapine derivative for optoelectronic semiconductors is 10,10″-bis(2-ethylhexyl)-2,2″,7,7″-tetraalkyl-10H, 10″H-dispiro[acridine-9,4′-dithieno[3,2-c:2′,3′-e]oxepine-6′,9″-acridine], having the formula:

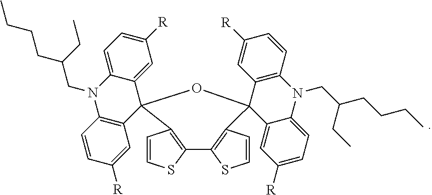

The at least one R₁, R₂, R₃, R₄, R₅, R₆, R₇, or R₈ substituent in the compounds according to any one of formulae (Ia), (Ib), (IIa), (IIb), (IIIa), and (IIIb), which is different from H, is independently selected from a substituent of formula (1):

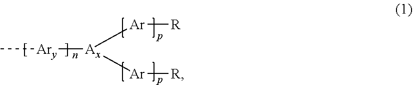

wherein n and p at least on integer selected from 0, 1, or 2; A_{X} is selected from N or P(═O), and is preferable N; Ar_{y} and Ar are independently selected from a monocyclic system or a polycyclic system comprising fused aromatic rings or conjugated monocyclic aromatic rings, said ring comprising 0, 1, or 2 heteroatoms selected from O, S, and N, and is further substituted, in addition to R, by other substituents independently selected from H, halogen, C1-C10 alkyl, C1-C10 alkoxy group, C1-C10 alkylthio (—S-alkyl), and —C≡N; and R is selected from H, R₁, —NR₁R₂, —O—R₂, —P(═O)R₁R₂, —S—R₁, or halogen, wherein R₁ and R₂ are independently selected from C4-C20 aryl, C4-C20 heteroaryl, C4-C20 aryloxy group, C4-C20 heteroaryloxy group, C4-C20 alkoxyaryl, C4-C20 alkoxyheteroaryl, C4-C20 aryl aryloxy group, C4-C20 heteroaryl aryloxy group, C1-C20 alkyl, C1-C20 alkoxy group, C1-C20 alkoxyalkyl, C1-C20 alkylthio, C2-C20 alkenyl and C2-C20 alkynyl, wherein said alkyl, alkoxy, alkoxyalkyl, alkenyl and alkynyl, if they comprise 3 or more carbons, may be linear, branched or cyclic, and wherein aryl, heteroaryl, alkyl, alkenyl, alkynyl may be further substituted by alkoxy group, alkylthio group and alkyl.

The dotted line in formula (1) represents the bond by which Ar_{y} (if Ar_{y} is present and n is 1 or 2) or A_{X} (if Ar_{y} is absent) is connected to the aromatic ring of the fused ring system. Preferably, n is 0 or 1 and p is 1 or 2. If A_{X} is N, preferably n is 0 or 1 and p is 1 or 2. If A_{X} is P(═O), preferably n is 1 and p is 1 or 2. The Ar_{y} (Ar_{y1} and Ar_{y2}) and Ar moieties (Ar₁ and Ar₂) may be identical or different. If A_{X} is N, preferably n is 0 or 1, p is 1 or 2, and Ar_{y1} (if present), Ar₁ and Ar₂ (if present) are identical. If A_{X} is P(═O), preferably n is 1, p is 1 or 2, Ar_{y1}, Ar₁ and Ar₂ (if present) are identical or different, preferably identical. A_{X} may be N.

Ar_{y} and Ar of formula (1) may be independently selected from moieties according to any one of formulae (2) to (19) as follows:

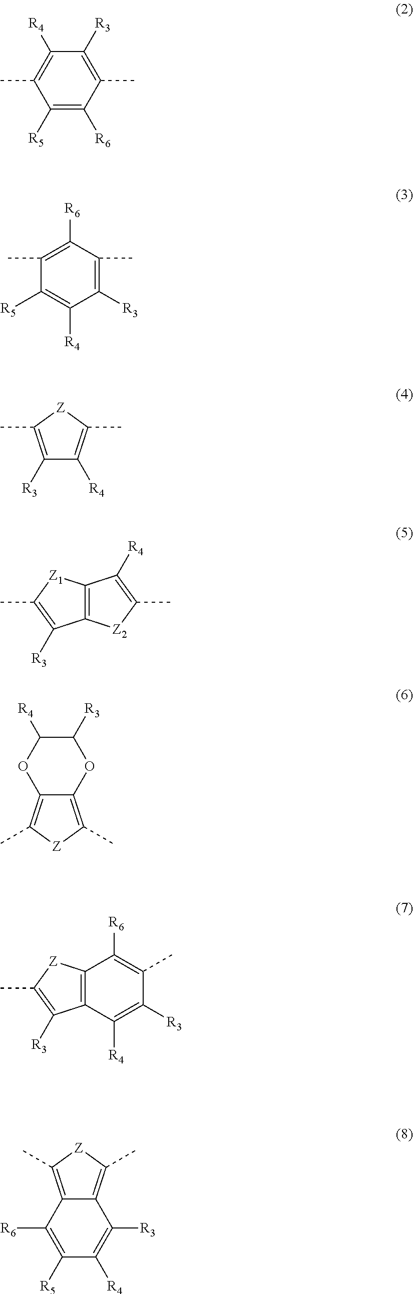

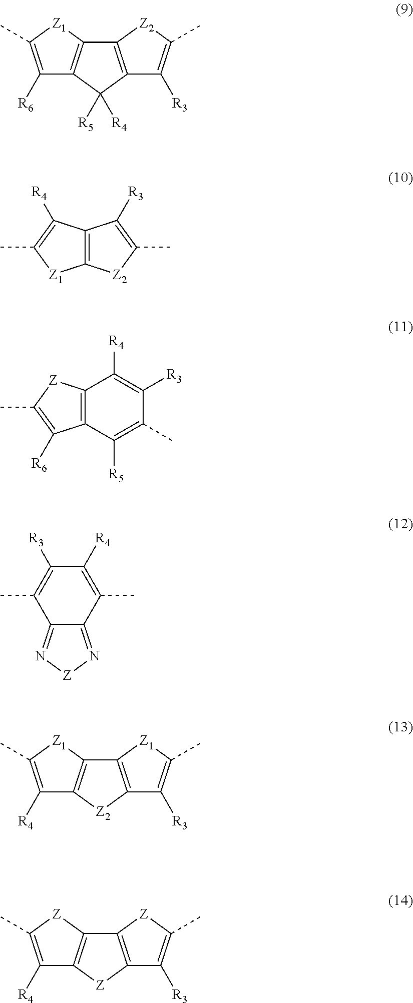

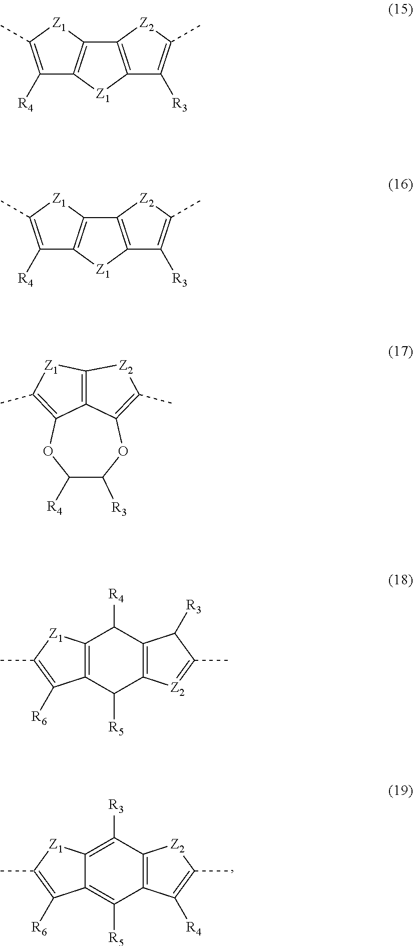

wherein Z, Z₁, and Z₂ are independently selected from O, S, and Se atoms, and Z₁ is different from Z₂ when they are present in the same moiety; and R₃, R₄, R₅, and R₆ are independently selected from H, halogen, C1-C10 alkyl, C1-C10 alkoxy group, C1-C10 alkylthio (—S-alkyl), and —C≡N.

The dotted lines represent the bond by which the substituents are connected to the aromatic ring of the fused ring system and/or to another substituent and/or to the N atom and/or to the P(═O).

Z, Z₁, and Z₂ preferably are independently selected from O and S. Preferably, Ar_{y} and Ar are independently selected from moieties according to any one of formulae (2), (3), (4), (6), (13), and (14), and more preferably from moieties according to any one of formulae (2), (3), and (4), preferably (2) and (3), in particular when n is 1 and p is 2.

R₉, R₁₀, R₁₁, R₁₂, R₁₃, R₁₄, R₁₅, R₁₆, R₁₇, and R₁₈ may be independently selected from H, halogen selected from Cl, F, Br, or I, C1-C30 alkyl, C1-C30 heteroalkyl, C4-C20 aryl, C4-C20 heteroaryl, C4-C30 alkylaryl group, C4-C30 aryloxy group or C4-C20 heteroaryloxy group, wherein the heteroatom is selected from O, S, N, —P(═O)—, —C≡N, preferably from O, S, and N, more preferably from O and S, and wherein alkyl, heteroalkyl, alkylaryl, if they comprise 3 or more carbons, may be linear, branched or cyclic, and from a substituent of formula (1) as defined above.

R₉, R₁₀, R₁₁, R₁₂, R₁₃, R₁₄, R₁₅, R₁₆, R₁₇, and R₁₈ may be independently selected from H and from a substituent of formula (1), as defined above.

The compound of formula (I) may be selected from a moiety of formula (K1a) or (K2a), as follows:

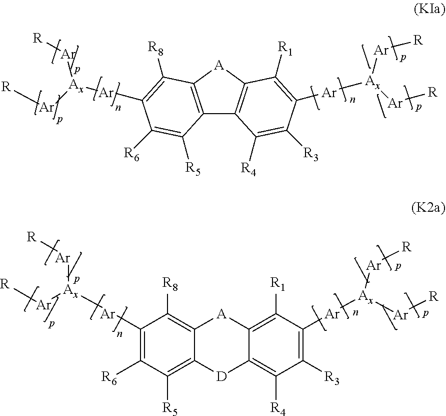

wherein A, Ar_{y}, A_{X}, Ar, n, p, R, R₁, R₂, R₃, R₄, R₅, R₆, R₇, R₈, R₉, R₁₀, R₁₁, R₁₂, R₁₃, R₁₄, R₁₅, R₁₆, R₁₇, and R₁₈ are defined as above.

The compound according to formula (Ia) may have 2 moieties of formula (K1a), and the compound according formula (Ib) may have 2 moieties of formula (K2a). Both formula (Ia) and (Ib) may contain two moieties of K1 or K1a or K2 or K2a, and may be the same or may consist of one K1 or K1a and one K2 or K2a or a combination. In general, the two moieties are preferably the same.

Any of the unsubstituted R₁-R₈ by formula (1) of the compound of the present subject matter according to any one of formulae (Ia), (Ib), (IIa), (IIb), (IIIa), and (IIIb) may be H.

R₉, R₁₀, R₁₁, R₁₂, R₁₃, R₁₄, R₁₅, R₁₆, R₁₇, and R₁₈ of a compound according to any one of formulae (Ia) and (Ib) may be H. In particular, R₉, R₁₀, R₁₁, R₁₂, R₁₃, R₁₄, R₁₅, R₁₆, R₁₇, and R₁₈ of a compound selected from a compound according to any one of formulae (K1a) and (K2a) may be H.

The dispiro-oxepine/dispiro-thiapine derivatives for optoelectronic semiconductors provide a hole transporting material comprising at least one compound selected from formulae (Ia), (Ib), (IIa), (IIb), (IIIa), and (IIIb), as defined above.

The dispiro-oxepine/dispiro-thiapine derivatives for optoelectronic semiconductors may be used in an optoelectronic and/or photoelectrochemical device comprising at least one compound selected any one of formulae (Ia), (Ib), (IIa), (IIb), (IIIa), and (IIIb), as defined above. The optoelectronic and/or photoelectrochemical device may have a hole transporting layer comprising said at least one compound selected from a compound according to any one of formulae (Ia), (Ib), (IIa), (IIb), (IIIa), and (IIIb), as defined above.

The optoelectronic and/or photoelectrochemical device may be an organic photovoltaic device, a photovoltaic solid-state device, a p-n heterojunction, an organic solar cell, a dye-sensitized solar cell, a solid-state solar cell, a phototransistor, a photodetector, a particle detector, or an OLED (organic light-emitting diode). In particular, the optoelectronic and/or photoelectrochemical device may be a solar cell, a solid-state solar cell, or a photovoltaic solid state device and optical sensors.

A perovskite or organic solar cell using the dispiro-oxepine/dispiro-thiapine derivative for optoelectronic semiconductors typically comprises (1) a substrate, (2) a transparent electrode, and (3) an electron-transporting material infiltrated with a perovskite absorbing material; (4) a compound of the formulae (Ia), (Ib), (IIa), (IIb), (IIIa), and (IIIb) as hole transporting material coated on the electron transporting material; and (5) a counter electrode.

The optoelectronic and/or photoelectrochemical device may have a conducting support layer, a surface-increasing scaffold structure, an n-type semiconductor, a light-harvester layer or a sensitizer layer, a hole transporting layer, and a counter electrode and/or metal layer. The metal layer may be doped, as well as the n-type semiconductor. A conductive layer comprising a conductive material may be present between the hole transporting layer and the counter electrode and/or metal layer. The hole transporting layer may be provided on the sensitizer layer and is between the sensitizer layer and the conducting current providing layer, if present, or the counter electrode and/or metal layer. Further layers may be present.

The optoelectronic and/or photoelectrochemical device may comprise a combination of two or more compounds of the dispiro-oxepine/dispiro-thiapine derivatives for optoelectronic semiconductors as hole transporting material. The hole transporting layer may comprise the combination of two or more compounds.

The optoelectronic and/or photoelectrochemical device may comprise a hole collector layer, a conductive layer, an electron blocking layer, a sensitizer layer and a current collector layer, wherein the hole collector layer is coated by the conductive layer; and wherein the electron blocking layer is between the conductive layer and the sensitizer layer, which is in contact with the current collector layer being a metal or a conductor. The hole collector layer comprises a hole transporting material comprising at least one compound of the of the dispiro-oxepine/dispiro-thiapine derivatives for optoelectronic semiconductors according to any one of formulae (Ia), (Ib), (IIa), (IIb), (IIIa), and (IIIb).

The substrate can be made of glass, such as low-cost soda glass of high strength or non-alkali glass from which no alkaline elution occurs.

The conductive material may be selected from one or more conductive polymers or one or more hole transporting materials. Examples of such materials may include poly(3,4-ethylenedioxythiophene):poly(styrenesulfonate) (PEDOT:PSS), poly(3,4-ethylenedioxythiophene):poly(styrenesulfonate):grapheme nanocomposite (PEDOT:PSS:graphene), poly(N-vinylcarbazole) (PVK) and sulfonated poly(diphenylamine) (SPDPA), preferably PEDOT:PSS, PEDOT:PSS:graphene and PVK, more preferably PEDOT:PSS. Other suitable conductive polymers may include polyaniline, polypyrrole, polythiophene, polybenzene, polyethylenedioxythiophene, polypropylenedioxy-thiophene, polyacetylene, and combinations of two or more of the aforementioned, for example. Alternatively, a transparent polymer film may be used, such as tetraacetyl cellulose (TAC), polyethylene terephthalate (PET), polyethylene naphthalate (PEN), syndiotactic polystyrene (SPS), polyphenylenesulfide (PPS), polycarbonate (PC), polyarylate (PAr), polysulfone (PSF), polyestersulfone (PES), polyimide (Pl), polyetherimide (PEI), polycycloolefin such as polynorbornene, or brominated phenoxy resin. Polymer films are preferred, in particular PET, PEN, and polynorbornene.

The conducting support layer is preferably substantially transparent. “Transparent” means transparent to at least a part, preferably a major part, of the visible light. Preferably, the conducting support layer is substantially transparent to all wavelengths or types of visible light. Furthermore, the conducting support layer may be transparent to non-visible light, such as UV and IR radiation.

The conducting support layer may provide the support layer of a photovoltaic solid-state device. Preferably, the optoelectronic and/or electrochemical device is built on the support layer. The support of the device may be also provided on the side of the counter electrode. In this case, the conductive support layer does not necessarily provide the support of the device, but may simply be or comprise a current collector, for example, a metal foil.

The conducting support layer preferably functions and/or comprises a current collector, collecting the current obtained from the device. The conducting support layer may comprise a material selected from indium-doped tin oxide (ITO), fluorine-doped tin oxide (FTO), ZnO—Ga₂O₃, ZnO—Al₂O₃, tin oxide, antimony-doped tin oxide (ATO), SrGeO₃ and zinc oxide, preferably coated on a transparent substrate, such as plastic or glass. In this case, the plastic or glass provides the support structure of the layer, and the cited conducting material provides the conductivity. Such support layers are generally known as conductive glass and conductive plastic, respectively, which are thus preferred conducting support layers. The conducting support layer comprises a conducting transparent layer, which may be selected from conducting glass and from conducting plastic.

Suitable inorganic electron-transport materials are semi-conductive metal oxides, including oxides of titanium, tin, zinc, iron, tungsten, zirconium, hafnium, strontium, indium, cerium, yttrium, lanthanum, vanadium, cesium, niobium or tantalum. Furthermore, oxide-based semiconductors, such as M¹ₓM²_{y}O_{z} may be used, wherein M¹ and M² are, independently of each other, a metal atom, O is an oxygen atom, and x, y, and z are numbers (including 0). Examples are TiO₂, SnO₂, Fe₂O₃, WO₃, ZnO, Nb₂O₅, SrTiO₃, Ta₂O₅, Cs₂O, and zinc stannate. These semiconducting metal oxides can act as a scaffold structure in the solar cell.

The surface-increasing scaffold structure is provided on the conducting support structure or on a protective layer that may be provided on the scaffold structure. The surface-increasing scaffold structure is nanostructured and/or mesoporous.

The scaffold structure is made from and/or comprises a metal oxide. For example, the material of the scaffold structure is selected from semiconducting materials, such as Si, TiO₂, SnO₂, ZrO₂, Al₂O₃, Fe₂O₃, ZnO, WO₃, Nb₂O₅, CdS, ZnS, PbS, Bi₂S₃, CdSe, CdTe, SrTiO₃, GaP, InP, GaAs, CuInS₂, CuInSe₂, and combinations thereof. Preferred semiconductor materials are Si, TiO₂, SnO₂, ZnO, WO₃, Nb₂O₅, and SrTiO₃, for example.

There may be one or more intermediate layers between the scaffold structure and the conductive support. Such intermediate layers, if present, would preferably be conducting and/or semiconducting.

The sensitizer layer of the optoelectronic and/or photoelectrochemical device comprises at least one pigment, which may be organic, inorganic, organometallic and organic-inorganic pigments, or a combination thereof. The sensitizer is preferably a light absorbing compound or material. Preferably, the sensitizer is a pigment, and most preferably the sensitizer is an organic-inorganic pigment.

The sensitizer layer or light-harvester layer may comprise one or more pigments of the group consisting of organometallic sensitizing compounds (phthalocyanine derived compounds, porphyrine derived compounds), metal-free organic sensitizing compounds (diketopyrrolopyrrole (DPP)-based sensitizer), inorganic sensitizing compounds such as quantum dots, Sb₂S₃(Antimony sulfide, for example in the form of thin films), aggregates of organic pigments, nanocomposites, in particular, organic-inorganic perovskites, and combinations of the aforementioned.

The optoelectronic and/or photoelectrochemical device may be selected from a photovoltaic solid-state device or a solar cell comprising an organic-inorganic perovskite as sensitizer under the form of a layer. The perovskite structure has the general stoichiometry WMX₃, where “W” and “M” are cations, and “X” is an anion. The “W” and “M” cations can have a variety of charges, and in the original Perovskite mineral (CaTiO₃), the W cation is divalent and the M cation is tetravalent.

The light-harvester layer or the sensitizer layer may comprise, or consist of, or be made of an organic-inorganic perovskite. The organic-inorganic perovskite is provided under a film of one perovskite pigment or mixed perovskite pigments or perovskite pigments mixed with further dyes or sensitizers. The sensitizer layer may comprise a further pigment in addition to the organic-inorganic perovskite pigment, the further pigment selected from an organic pigment, an organometallic pigment, or an inorganic pigment. The perovskite formulae may include structures having three (3) or four (4) anions, which may be the same or different, and/or one or two (2) organic cations, and/or metal atoms carrying two or three positive charges, in accordance with the formulae presented elsewhere herein.

The organic-inorganic perovskite layer material may comprise a perovskite-structure according any one of formulae (IV), (IVa), (IVb), (IVc), (IVd), and (IVe) below:

WW′MX₄ (IV),

WMX₃ (IVa),

WW′N_{2/3}X₄ (IVb),

WN_{2/3}X₃ (IVc),

BN_{2/3}X₄ (IVd),

BMX₄ (IVe),

wherein W and W′ are organic, monovalent cations independently selected from primary, secondary, tertiary, or quaternary organic ammonium compounds, including N-containing heterorings and ring systems, W and W′ having independently from 1-60 carbons and 1-20 heteroatoms; B is an organic, bivalent cation selected from primary, secondary, tertiary, or quaternary organic ammonium compounds having from 1-60 carbons and 2-20 heteroatoms and having two positively charged nitrogen atoms; M is a divalent metal cation selected from Cu²⁺, Ni²⁺, Co²⁺, Fe²⁺, Mn²⁺, Cr²⁺, Pd²⁺, Cd²⁺, Ge²⁺, Sn²⁺, Pb²⁺, Eu²⁺, or Yb2+; N is selected from Bi³⁺ and Sb³⁺; and X is independently selected from Cl⁻, Br⁻, I⁻, NCS⁻, CN—, BF₄⁻, PF₆⁻, CNO⁻, SeCN⁻, and NCO⁻. In particular, each X may be identical or different. For example, in AMX₃ (formula IIa) may be expressed as formula (IVa′) below:

WMXiXiiXiii (IVa′),

wherein Xi, Xii, and Xiii are independently selected from Cl⁻, Br⁻, I⁻, NCS⁻, CN⁻, and NCO⁻, preferably from halides (Cl⁻, Br⁻, I⁻); and W and M are as defined above.

Thus, Xi, Xii, and Xiii may be the same or different. Preferably, if Xi, Xii, and Xiii in formulae (IVa) and (IVc), or Xi, Xii, Xiii, and Xiv in formulae (IV), (IVb), (IVd), or (IVe), comprise different anions X, there are not more than two different anions. For example, Xi and Xii would be the same, and Xiii would be an anion that is different from Xi and Xii.

The organic-inorganic perovskite layer may comprise a perovskite structure according to any one of the formulae (IVf) to (IVl):

WPbX₃ (IVf),

WSnX₃ (IVg).

WBiX₄ (IVh),

WW′PbX₄ (IVi),

WW′SnX₄ (IVj),

BPbX₄ (IVk),

BSnX₄ (IVl),

wherein W, W′, B and X are as defined above. Preferably, X is selected from Cl⁻, Br⁻, and I⁻, and most preferably, X is I⁻ or a mixture of Br⁻ and I⁻.

The organic-inorganic perovskite layer may comprise a perovskite structure of the formulae (IVf) to (IVl), more preferably (IVf) and/or (IVg) above.

W and W′ may be monovalent cations selected independently from any one of the compounds of formulae (20) to (28) below:

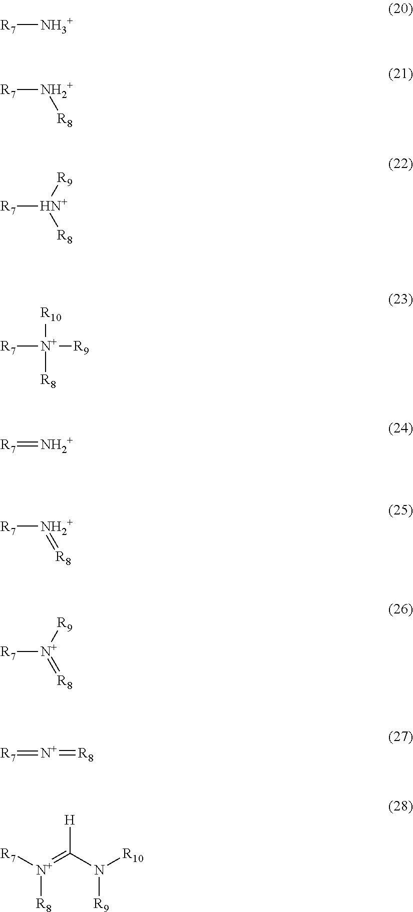

wherein, R₇, R₈, R₉, and R₁₀ are independently selected from C1-C15 organic substituents comprising from 0-15 heteroatoms. In the C1-C15 organic substituent, any one, several, or all hydrogens in the substituent may be replaced by halogen, and the organic substituent may comprise up to fifteen (15) N, S, or O heteroatoms, and in any one of the compounds (20) to (28), two or more of the substituents present (R₇, R₈, R₉, and R₁₀, as applicable) may be covalently connected to each other to form a substituted or unsubstituted ring or ring system. Preferably, in a chain of atoms of the C1-C15 organic substituent, any heteroatom is connected to at least one carbon atom. Preferably, neighboring heteroatoms are absent and/or heteroatom-heteroatom bonds are absent in the C1-C15 organic substituent comprising from 0 to 15 heteroatoms. The heteroatoms may be selected from N, S, and/or O.

R₇, R₈, R₉, and R₁₀ may be independently selected from C1 to C15 aliphatic and C4 to C15 aromatic or heteroaromatic substituents, wherein any one, several, or all hydrogens in the substituent may be replaced by halogen, and wherein in any one of the compounds (20) to (28), two or more of the substituents present may be covalently connected to each other to form a substituted or unsubstituted ring or ring system.

The organic-inorganic perovskite in the device may be selected from a compound of formula (IV) or (IVa).

B may be a bivalent cation selected from any one of the compounds of formulae (29) and (30) below:

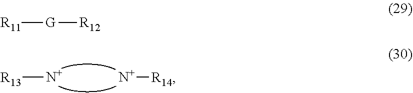

wherein, in the compound of formula (29), G is an organic linker structure having 1 to 10 carbons and 0 to 5 heteroatoms selected from N, S, and/or O, wherein one or more hydrogen atoms in G may be replaced by halogen; and R₁₁ and R₁₂ are independently selected from a compounds of any one of formulae (20) to (28); and wherein in the compound of formula (30), the circle containing the two positively charged nitrogen atoms represents a substituted or unsubstituted aromatic ring or ring system comprising 4-15 carbon atoms and 2-7 heteroatoms or 4-10 carbon atoms and 2-5 heteroatoms, wherein said nitrogen atoms are ring heteroatoms of a ring or ring system, and wherein the remaining heteroatoms may be selected independently from N, O and S, and wherein R₁₃ and R₁₄ are independently selected from H and from a compound of any one of formulae (20) to (28). Halogen atom substituting hydrogen atom totally or partially may also be present in addition to and/or independently of the 2-7 heteroatoms.

Preferably, if the number of carbons in G is impaired, the number of heteroatoms is smaller than the number of carbons. Preferably, in the ring structure of formula (30), the number of ring heteroatoms is smaller than the number of carbon atoms. G may be an aliphatic, aromatic or heteroaromatic linker structure having from 1-10 carbons.

R₇, R₈, R₉, and R₁₀ may be independently selected from C1 to C10 alkyl, C2 to C10 alkenyl, C2 to C10 alkynyl, C4 to C10 heteroaryl and C6 to C10 aryl, wherein said alkyl, alkenyl, and alkynyl, if they comprise 3 or more carbons, may be linear, branched or cyclic, wherein said heteroaryl and aryl may be substituted or unsubstituted, and wherein several or all hydrogens in R₇, R₈, R₉, and R₁₀ may be replaced by halogen.

Alternatively, R₇, R₈, R₉, and R₁₀ may be independently selected from C1 to C8 alkyl, C2 to C8 alkenyl, C2 to C8 alkynyl, C4 to C8 heteroaryl and C6 to C8 aryl, wherein said alkyl, alkenyl, and alkynyl, if they comprise 3 or more carbons, may be linear, branched or cyclic, wherein said heteroaryl and aryl may be substituted or unsubstituted, and wherein several or all hydrogens in R₇, R₈, R₉, and R₁₀ may be replaced by halogen.

Alternatively, R₇, R₈, R₉, and R₁₀ may be independently selected from C1 to C6 alkyl, C2 to C6 alkenyl, C2 to C6 alkynyl, C4 to C6 heteroaryl and C6 aryl, wherein said alkyl, alkenyl, and alkynyl, if they comprise 3 or more carbons, may be linear, branched or cyclic, wherein said heteroaryl and aryl may be substituted or unsubstituted, and wherein several or all hydrogens in R₇, R₈, R₉, and R₁₀ may be replaced by halogen.

Alternatively, R₇, R₈, R₉, and R₁₀ may be independently selected from C1 to C4 alkyl, C2 to C4 alkenyl and C2 to C4 alkynyl, wherein said alkyl, alkenyl and alkynyl, if they comprise 3 or more carbons, may be linear, branched or cyclic, and wherein several or all hydrogens in R₇, R₈, R₉, and R₁₀ may be replaced by halogen.

Alternatively, R₇, R₈, R₉, and R₁₀ may be independently selected from C1 to C3, preferably C1 to C2 alkyl, C2 to C3, preferably C2 alkenyl and C2 to C3, preferably C2 alkynyl, wherein said alkyl, alkenyl and alkynyl, if they comprise 3 or more carbons, may be linear, branched or cyclic, and wherein several or all hydrogens in R₇, R₈, R₉, and R₁₀ may be replaced by halogen.

Alternatively, R₇, R₈, R₉, and R₁₀ may be independently selected from C1 to C4, more preferably C1 to C3, and even more preferably C1 to C2 alkyl. Most preferably R₇, R₈, R₉, and R₁₀ are methyl. Again, the alkyl may be completely or partially halogenated.

W, W′, and B may be monovalent (W, W′) and bivalent (B) cations, respectively, selected from substituted and unsubstituted C5 to C6 rings comprising one, two, or more nitrogen heteroatoms, wherein one (for W and W′) or two (for B) of the nitrogen atoms is/are positively charged. Substituents of such rings may be selected from halogen and from C1 to C4 alkyl, C2 to C4 alkenyl and C2 to C4 alkynyl, as defined above, preferably from C1 to C3 alkyl, C3 alkenyl, and C3 alkynyl, as defined above. The ring may comprise further heteroatoms, which may be selected from O, N, and S. Bivalent organic cations B comprising two positively charged ring N-atoms are exemplified, for example, by the compound of formula (30) above. Such rings may be aromatic or aliphatic.

W, W′, and B may also comprise a ring system having two or more rings, at least one of which is a substituted or unsubstituted C5 to C6 ring, as defined above. The elliptically drawn circle in the compound of formulae (30) may also represent a ring system having, for example, two or more rings, but preferably two rings. Also, if W and/or W′ comprises two rings, further ring heteroatoms may be present, which are preferably not charged, for example.

However, the organic cations W, W′, and B may comprise one (for W, W′), two (for B), or more nitrogen atom(s), but are free of any O or S or any other heteroatom, with the exception of halogens, which may substitute one or more hydrogen atoms in cation W and/or B.

W and W′ preferably comprise one positively charged nitrogen atom. B preferably comprises two positively charged nitrogen atoms.

W, W′, and B may be selected from the exemplary rings or ring systems of formulae (31) and (32) (for W, W′) and from (33) to (35) (for B) below:

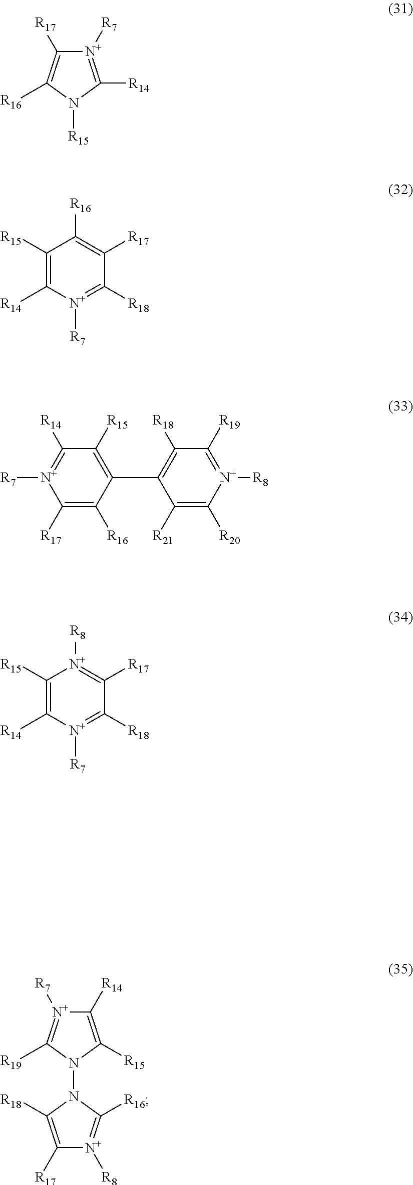

wherein R₇ and R₈ are selected from substituents as defined above; and R₁₄, R₁₅, R₁₆, R₁₇, R₁₈, R₁₉, R₂₀, and R₂₁ are independently selected from H, halogen and substituents as defined above for R₇, R₈, R₉, and R₁₀.

In an embodiment, preferably, R₁₄, R₁₅, R₁₆, R₁₇, R₁₈, R₁₉, R₂₀, and R₂₁ are selected from H and halogen, most preferably H.

In the organic cations W, W′, and B, hydrogen atoms may be substituted by halogens, such as F, Cl, I, and Br, preferably F or Cl. Such a substitution is expected to reduce the hygroscopic properties of the perovskite layer or layers and may thus provide a useful option, as described herein.

W and W′ may be independently selected from organic cations of formula (20) and/or formula (28). The metal M may be selected from Sn²⁺ and Pb²⁺, preferably Pb²⁺. N may be Sb³⁺. The three or four X may be independently selected from Cl⁻, Br⁻, and I⁻.

The dispiro-oxepine/dispiro-thiapine derivatives for optoelectronic semiconductors reduce triggering instability and improve the lifetime of a perovskite-containing device as compared to devices that use spiro-OMeTAD as the HTM (hole transport material). The present derivatives provide a molecularly engineered HTM for efficient PSC with a certified power conversion efficiency of over 20% that was synthesized with a simple dissymmetric fluorene-dithiophene core.

Photovoltaic technology is one of the most effective approaches to utilize solar energy, which directly converts sunlight into electricity. The dispiro-oxepine/dispiro-thiapine derivatives for optoelectronic semiconductors also provide a new hole transporting material allowing tuning of the HOMO level and having a positive impact on the sensitizer through its passivation to improve and provide higher PCE to photovoltaic devices comprising perovskite, as well as to other optoelectronic devices, for example, Organic Light Emitting Diodes (OLED) and Field Effect Transistors (FET).

The dispiro-oxepine/dispiro-thiapine derivatives for optoelectronic semiconductors provide an efficient solar cell, which can be rapidly prepared in an efficient way, using readily available or low-cost materials, such as conductive material, and using a short manufacturing procedure based on industrially known manufacturing steps, keeping the material costs and the material impact on the environment very low.

The dispiro-oxepine/dispiro-thiapine derivatives for optoelectronic semiconductors relate to certain organic compounds, as well as their use as hole transport materials. In this regard, these compounds may be used to tune HOMO levels in optoelectronic and/or electrochemical devices, such as lasing, light emitting devices, and can be used for photo detection, particularly in solar cells and solid-state solar cells, including tandem cell applications comprising these compounds. The dispiro-oxepine/dispiro-thiapine derivatives for optoelectronic semiconductors may be used as hole transporting material and may function as hole injection materials to bring holes extracted from a sensitizer to the hole collector of a photovoltaic device, e.g., a solid solar cell. The dispiro-oxepine/dispiro-thiapine derivatives for optoelectronic semiconductors are able to passivate the sensitizer or the sensitizer layer and to improve the performance and the efficiency of such a device, and in particular an optoelectronic and/or photoelectrochemical device comprising an organic-inorganic perovskite as sensitizer.

The dispiro-oxepine/dispiro-thiapine derivatives for optoelectronic semiconductors will now be illustrated by the following examples, which do not limit the scope defined by the appended claims.

### Example 1

### General Synthesis of Dispiro-Oxepine/Dispiro-Thiapine Derivatives

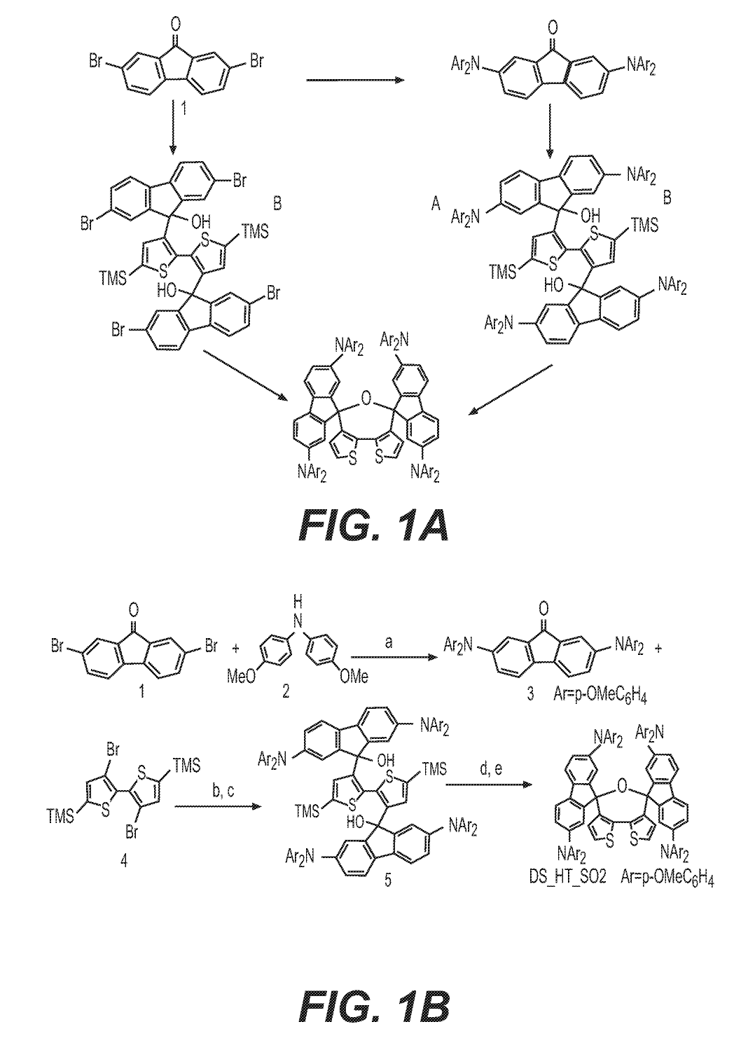

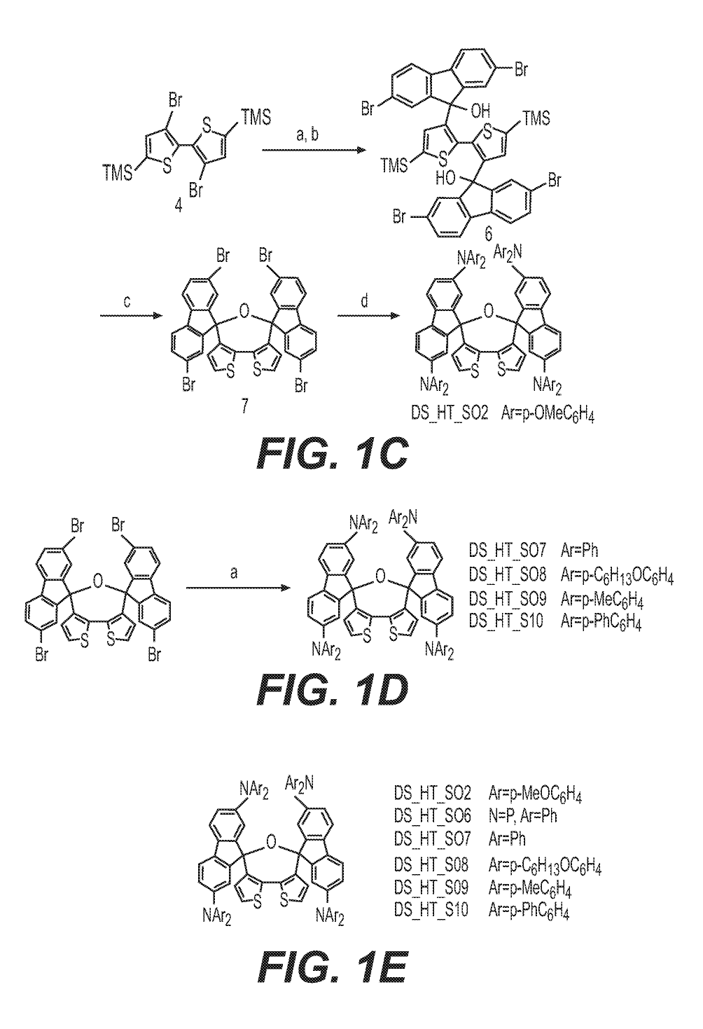

Spiro compounds DS-HT-SO2, DS-HT-SO7, DS-HT-SO8, DS-HT-SO9, and DS-HT-SO10, shown in FIG. 1E, comprise a two diarylamino functionalized fluorene moiety and a one dithiophene unit, which, together with a two sp³-hybridized carbon atom, form the seven-membered oxepine. Only a few examples of related dispiro compounds, devoid of electron-donating diarylamino substituents on the fluorene portion of the molecule, have been disclosed in the art so far, and synthesis has proved cumbersome. Indeed, considerable amounts of by-products are formed when the intramolecular ring closure leading to the formation of the spiro linkage is carried out on 2,2′-bithiophene derivatives under standard acidic conditions. Low yields after extensive purification of complex reaction mixtures have been reported. Recently, it has been shown that the introduction of protecting groups on the electron-rich α-positions of the thiophene units, in combination with the use of suitable Lewis acids, markedly increases the efficiency of the intramolecular cyclization process. Based on these considerations, two viable synthetic routes towards diarylamino functionalized fluorene-bithiophene dispiro compounds were developed by the present inventors, which differ in the stage of introduction of the diarylamino groups and are outlined in FIG. 1A.

Both strategies were explored in the case of DS-HT-SO2, and the synthetic details are summarized here. Following strategy A of FIG. 1A, and as shown more particularly in FIG. 1B, a palladium-catalyzed coupling reaction between 2,7-dibromofluoren-9-one 1 and bis(4-methoxyphenyl)amine 2 allowed the introduction of two diarylamino substituents onto the fluorene moiety at the first stage. 2,7-Bis(bis(4-methoxypheyl)amino)fluoren-9-one 3 was thus isolated in excellent yield. Subsequent reaction of 3 with the carbanion generated by treatment of 3,3-dibromo-5,5′-bis(trimethylsilyl)-2,2′-bithiophene 4 with BuLi at low temperature afforded the tertiary alcohol 5 in 75% yield. The subsequent cyclization of 5 carried out in acetic acid in the presence of a catalytic amount of hydrochloric acid that results in the formation of dispiro-oxepine derivative DS-HT-SO2 quantitatively.

The desired product DS-HT-SO2 could be readily isolated by column chromatography on silica in 66% yield (overall yield from 1=46.5%).

Following strategy B of FIG. 1A, and as shown more particularly in FIG. 1C, bithiophene derivative 4 was treated with BuLi at low temperature and then reacted with 2,7-dibromofluoren-9-one 1 to give carbinol 6 in 80% yield. Intramolecular cyclization mediated by acetic acid and a catalytic amount of hydrochloric acid was effective also for this derivative, affording the key intermediate 7 in 80% yield. Finally, the targeted compound DS-HT-SO2 was obtained by palladium-catalyzed reaction of 7 with bis(4-methoxyphenyl)amine 2 in 81% yield (overall yield from 1=51%).

Strategies A and B here described are both suitable for the preparation of the targeted compounds. The second strategy (B) seems to be preferable because the presence of two bromine atoms on the spiro compound 7 opens the way to a variety of promising structural modifications through well-established synthetic procedures. To explore this option, compound 7 was used as the common intermediate in the preparation of difluorene/bithiophene spiro derivatives by palladium-catalyzed amination reaction with different diarylamines, as shown in FIG. 1D. Compounds DS-HT-SO7, DS-HT-SO8, DS-HT-SO9 and DS-HT-SO10, shown in FIG. 1E, were obtained in this manner in good to excellent yields, ranging from 65 to 80%.

All available chemicals were purchased from commercial sources and were used without any further purification. Solvents were purified by standard methods and dried if necessary. 3-Bromo-5,5′-bis(trimethylsilyl)-2,2′-bithiophene 4 was prepared as previously described. Compound 2,7-Bis(bis(4-methoxypheyl)amino)-fluoren-9-one 3 was prepared according to a modification of a procedure reported in the literature. Para-substituted diarylamines were synthesized according to a 2-step procedure involving the Ulmann-like coupling of two equivalents of para-substituted aryliodide with BocNH₂, followed by deprotection of the Ar₂N-Boc intermediate under acidic conditions. Reactions were monitored by thin layer chromatography (TLC) that was conducted on plates precoated with silica gel Si 60-F254 (Merck, Germany). Column chromatography was conducted using silica gel Si 60, 0.063-0.200 mm (normal) or 0.040-0.063 mm (flash) (Merck, Darmstadt, Germany). ¹H and ¹³C NMR spectra were recorded on a Bruker Avance 400 (400 and 100.6 MHz, respectively); chemical shifts are indicated in parts per million downfield from SiMe₄, using the residual proton (CHCl₃=7.26 ppm) and carbon (CDCl₃=77.0 ppm) solvent resonances as the internal reference. Protons and carbon assignments were achieved by ¹³C-APT, ¹H-¹H COSY, and ¹H-¹³C heteronuclear correlation experiments. Coupling constant values J are given in Hz.

### Example 2

### Synthesis of DS-HT-SO2

The synthesis of DS-HT-SO2 is shown in FIG. 1C. To a solution of 5,5′-ditrimethylsilyl-3,3′-dibromo-2,2′-bithiophene 6 (0.5 g, 1.06 mmol) in 20 mL of dry THF at −78° C. under argon atmosphere, n-BuLi (2.5 M in hexanes, 0.95 mL, 2.3 mmol) was dropwise added. After 30 minutes at the same temperature, 2,7-dibromofluorenone 3 (469 mg, 1.39 mmol) in THF (10 mL) was added to the mixture dropwise. At the end of the addition, the cooling bath was removed and mixture was allowed to return to room temperature and left under stirring for overnight. The solution was hydrolyzed with water, extracted with diethyl ether, and the combined organic phases were dried over MgSO₄. The crude material was purified by flash column chromatography (silica gel, Hexane:CH₂Cl₂ 7:3) affording the title compound as a white solid. ¹H NMR (300 MHz, CDCl₃): 7.47-7.91 (m, 12H), 6.19 (s, 2H), 0.23 (s, 18H); HRMS (FAB): Calculated 985.8231, Found 985.8215.

The Brønsted acid-mediated cyclization of compound 6 was performed as follows. Carbinol compound 6 was dissolved in boiling acetic acid (200 mL), and several drops (around 1 ml) of concentrated hydrochloric acid were added. The mixture was refluxed for 2 hours and a white solid appeared in the solution. After cooling to room temperature, the solvent was removed at reduced pressure and the residue taken up in AcOEt and washed with water. The organic phase was dried over MgSO₄, filtered and the solvent removed under reduced pressure. The crude material was purified by column chromatography (silica gel, petroleum hexane:CHCl₃ 9:1) to give the title compound (250 mg, 44% yield) as a white solid of both steps.

NMR spectral data for the two samples were identical. ¹H NMR (400 MHz, CDCl₃): 7.45 (dd, 4H, J=8.1-1.5 Hz), 7.39 (d, 4H, J=8.1 Hz), 7.15 (d, 2H, J=5.1 Hz), 7.06 (d, 4H, J=1.5 Hz), 6.37 (d, 2H, J=5.1 Hz); ₁₃C NMR (75 MHz, CDCl₃): 151.1, 140.8, 137.5, 134.4, 132.4, 129.1, 128.9, 124.8, 122.2, 121.4, 88.1; MS (MALDI-TOF) [M+]: 823.9.

DS-HT-SO2 was obtained from compound 7 as follows. In a flame dried Schlenk tube the spiro derivative 7 (500 mg, 1 mmol), bis(4-methoxyphenyl)amine 2 (700 mg, 5 mmol) and Pd₂(dba)₃ (100 mg, 0.15 mmol) were introduced under inert atmosphere. The Schlenk tube was evacuated and backfilled with nitrogen three times. After the addition of toluene (30 mL) and X-Phos (100 mg, 0.3 mmol), NaO^{t}Bu (350 mg, 6 mmol) was added and the reactor was brought into an oil bath pre-heated at 110° C. The reaction mixture was stirred at this temperature overnight. After cooling to room temperature, the mixture was diluted with Et₂O and washed with water and brine. The organic phase was dried over MgSO₄, filtered and the solvent removed at reduced pressure. The crude material was purified by flash column chromatography (silica gel, hexane:THF 8:2) to give the title compound (300 mg, 82% yield) as a yellowish solid. ¹H NMR (400 MHz, CDCl₃): δ 7.44 (d, J=8.4 Hz, 4H, Fluor-H), 7.05 (d, J=5.2 Hz, 2H, Thioph-H), 6.88 (d, J=9.2 Hz, 8H, Ar—H), 6.82 (dd, J=8.4 Hz, J=2.0 Hz, 4H, Fluor-H), 6.69 (d, J=9.2 Hz, 8H, Ar—H), 6.51 (d, J=5.2 Hz, 2H, Thioph-H), 6.47 (d, J=2.0 Hz, 4H, Fluor-H), 3.74 (s, 12H, OCH₃); ¹³C NMR (110 MHz, CDCl₃+1% v/v NH₂NH₂.H₂O): δ 155.3, 154.9, 147.3, 146.1, 141.2, 138.2, 135.0, 125.6 (Ar—C), 125.2 (Thioph-C), 121.7 (Fluor-C), 121.3 (Thioph-C), 119.6 (Fluor-C), 116.9 (Fluor-C), 114.4 (Ar—C), 61.7, 55.5 (OCH₃).

### Example 3

### General Synthesis Procedure for DS-HT-SO2 Through DS-HT-SO 10 by the Amination of 7

A flame dried Schlenk tube was charged with 7 (1 mmol), diarylamine (5 mmol) and Pd₂(dba)₃ (4 mol %). The Schlenk tube was evacuated and backfilled with nitrogen three times. After the addition of toluene (6 mL) and X-Phos (8 mol %), NaO^{t}Bu (6 mmol) was added and the reactor was brought into an oil bath pre-heated at 110° C. The reaction mixture was stirred at this temperature overnight. After cooling to room temperature, the mixture was diluted with CH₂Cl₂ and washed with water and brine. The organic phase was dried over MgSO₄, filtered, and the solvent removed at reduced pressure. The crude material was purified by column chromatography on silica gel.

### Example 4

### Synthesis of Solar Cell Having Mixed Hole Transport Materials

Nippon Sheet Glass 10 Ω/sq was cleaned by sonication in 2% Hellmanex water solution for 30 minutes. After rinsing with deionized water and ethanol, the substrates were further cleaned with UV ozone treatment for 15 minutes. Then, 30 nm TiO₂ compact layer was deposited on FTO via spray pyrolysis at 450° C. from a precursor solution of titanium diisopropoxide bis(acetylacetonate) in anhydrous ethanol. After the spraying, the substrates were left at 450° C. for 45 minutes and left to cool down to room temperature. Then, a mesoporous TiO₂ layer was deposited by spin coating for 20 s at 4000 rpm with a ramp of 2000 rpm s⁻¹, using 30 nm particle paste (Dyesol 30 NR-D) diluted in ethanol to achieve a 150-200 nm thick layer. After the spin coating, the substrates were immediately dried at 100° C. for 10 minutes and then sintered again at 450° C. for 30 minutes under dry air flow. Li-doping of mesoporous TiO₂ is accomplished by spin coating a 0.1 M solution of Li-TFSI in acetonitrile at 3000 rpm for 30 seconds, followed by another sintering step at 450° C. for 30 minutes. After cooling down to 150° C. the substrates were immediately transferred in a nitrogen atmosphere glove box for depositing the perovskite films.

The perovskite films were deposited from a precursor solution containing FAI (1 M) (formamidinium iodide), PbI₂ (1.1 M), MABr (0.2 M) (methylammonium iodide) and PbBr₂ (0.2 M) in anhydrous DMF:DMSO 4:1 (v:v). The perovskite solution was spin coated in a two-step program at 1000 and 4000 rpm for 10 and 30 seconds, respectively. During the second step, 100 μL of chlorobenzene was poured on the spinning substrate 15 seconds prior the end of the program. This perovskite is referred to as the “mixed perovskite”.

Another perovskite type, referred to as “standard perovskite, was prepared by dissolving a stoichiometric amount (1:1 molar ratio) of lead iodide and methyl ammonium iodide in dimethylsulfoxide at a concentration of 1.2 M of each component.

The substrates were then annealed at 100° C. for 1 hour in a nitrogen filled glove box.

After the perovskite annealing the substrates were cooled down for few minutes and a spirofluorene-linked methoxy triphenylamines (spiro-OMeTAD, Merck) solution (70 mM in chlorobenzene) was spin coated at 4000 rpm for 20 seconds. The spiro-OMeTAD was doped with bis(trifluoromethylsulfonyl)imide lithium salt (Li-TFSI, Sigma-Aldrich), tris(2-(1H-pyrazol-1-yl)-4-tert-butylpyridine)-cobalt(III) tris(bis(trifluoromethylsulfonyl)imide) (FK209, Dynamo) and 4-tert-Butylpyridine (TBP, Sigma-Aldrich). The molar ratio of additives for spiro-OMeTAD wa: 0.5, 0.03, and 3.3 for Li-TFSI, FK209, and TBP, respectively.

SO solutions were prepared with molarities ranging from 50-150 mM (in chlorobenzene). The optimized molar ratio of additives for SO₂ was: 0.5, 0.03, and 3.3 for Li-TFSI, FK209, and TBP, respectively. The SO solution was spin coated at 4000 rpm for 20 seconds. Finally, 70-80 nm of gold top electrode was thermally evaporated under high vacuum.

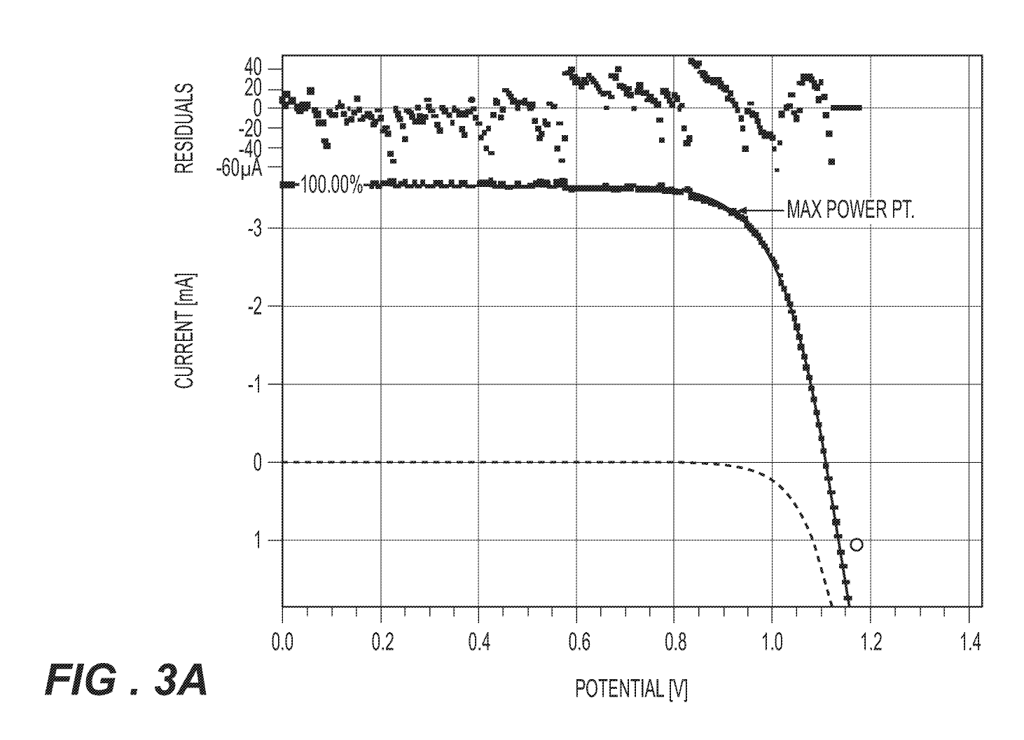

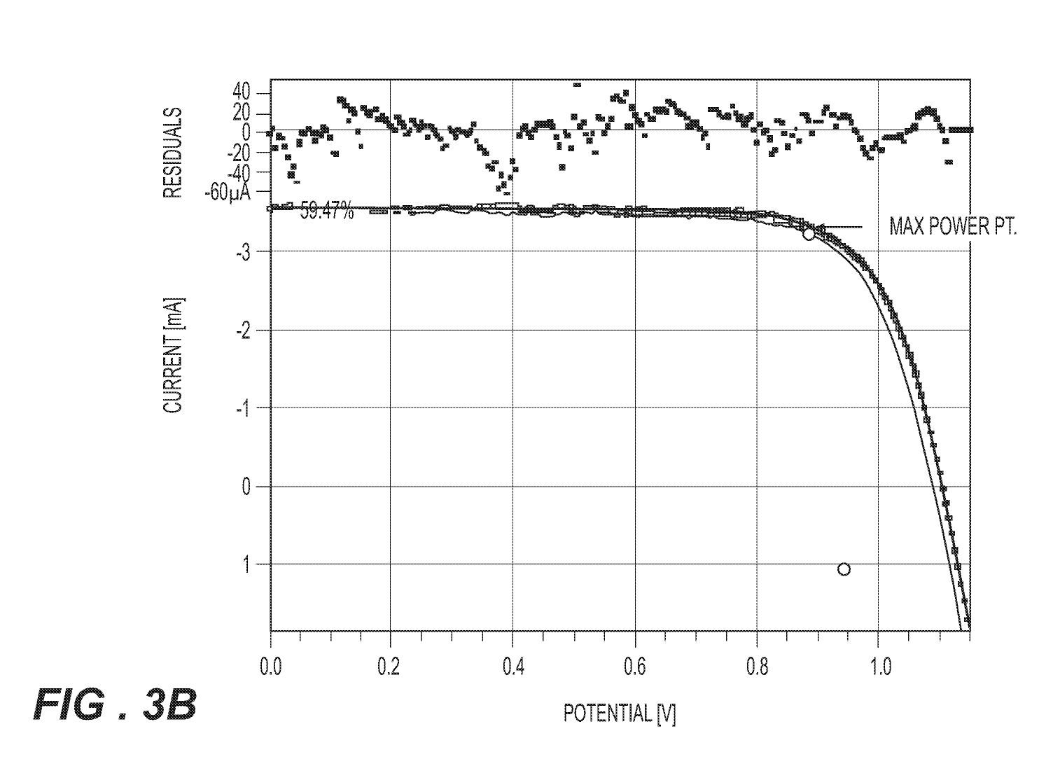

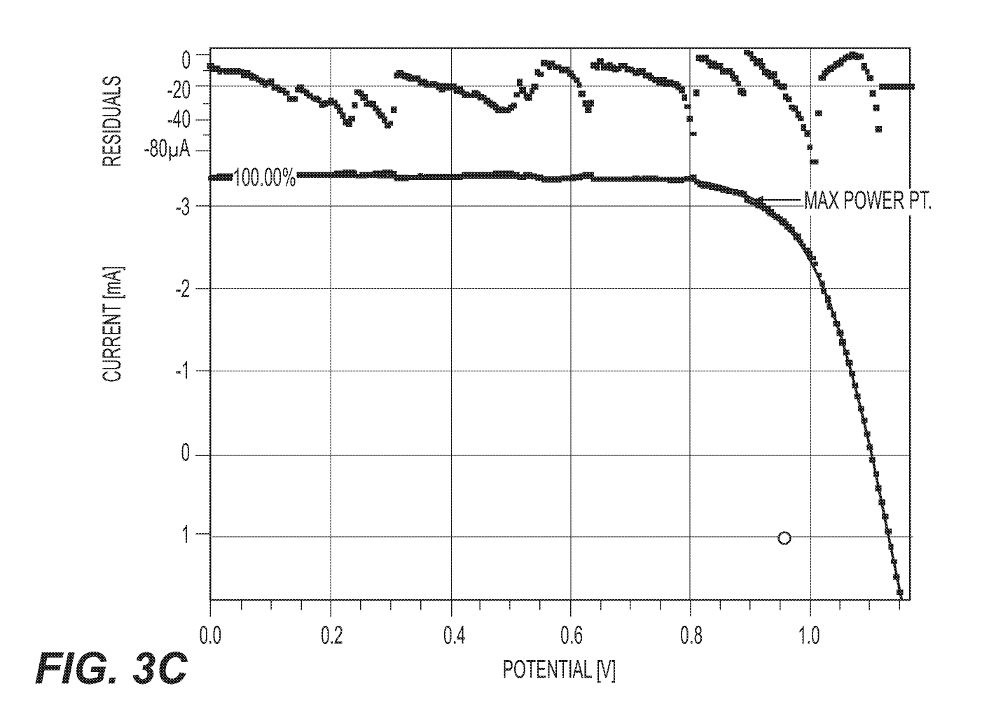

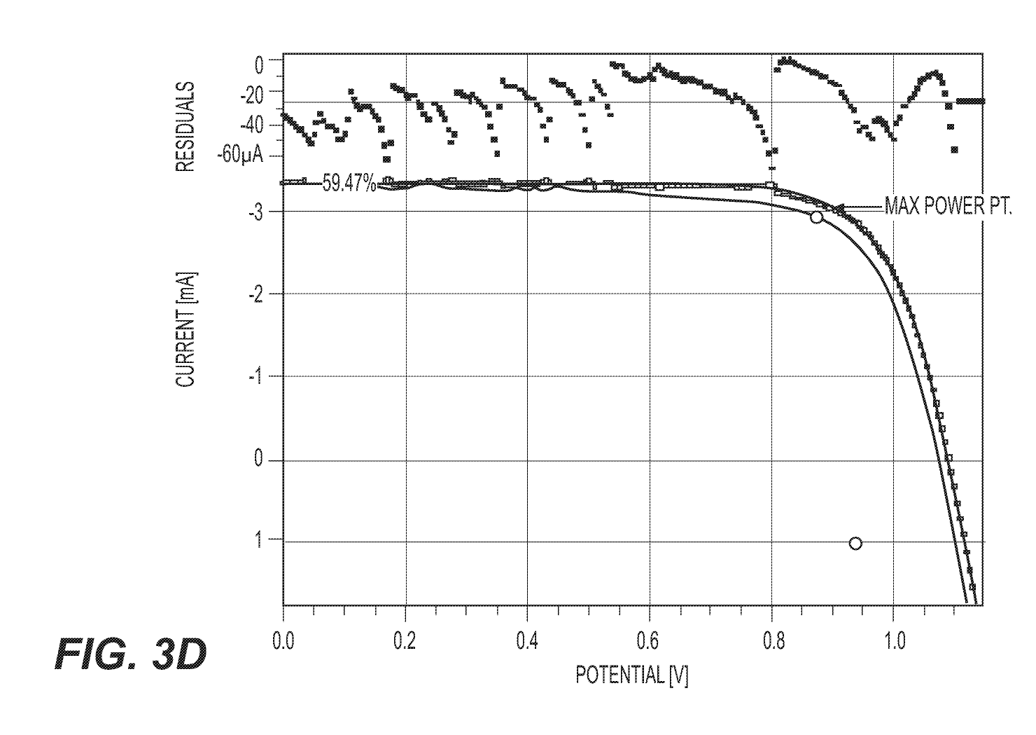

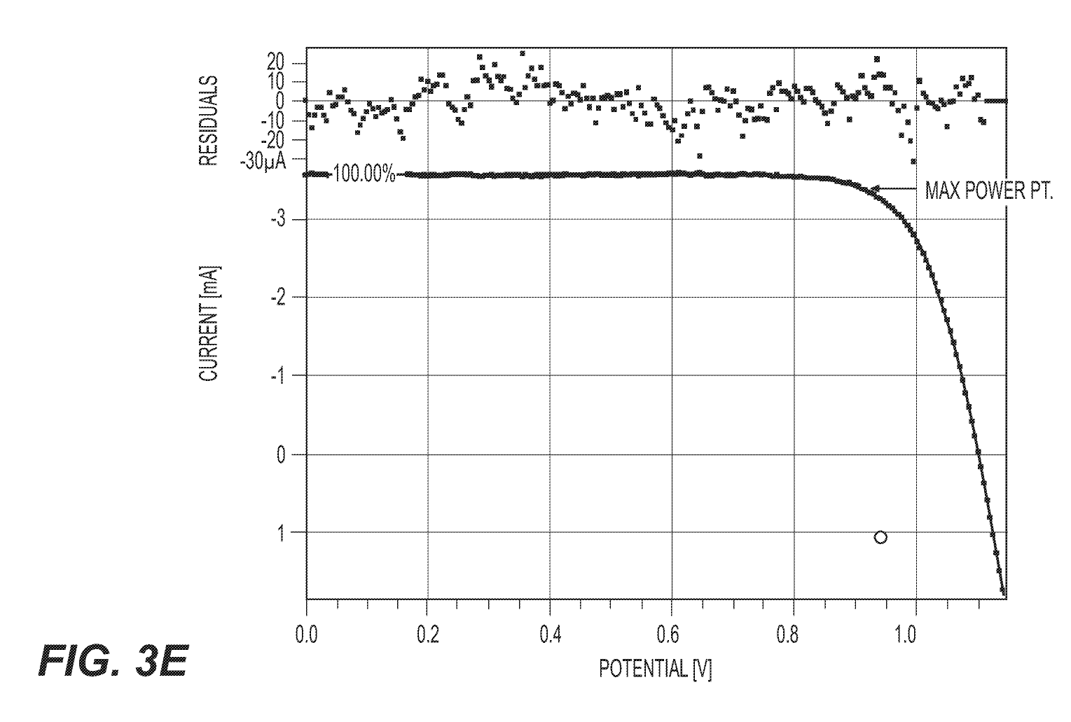

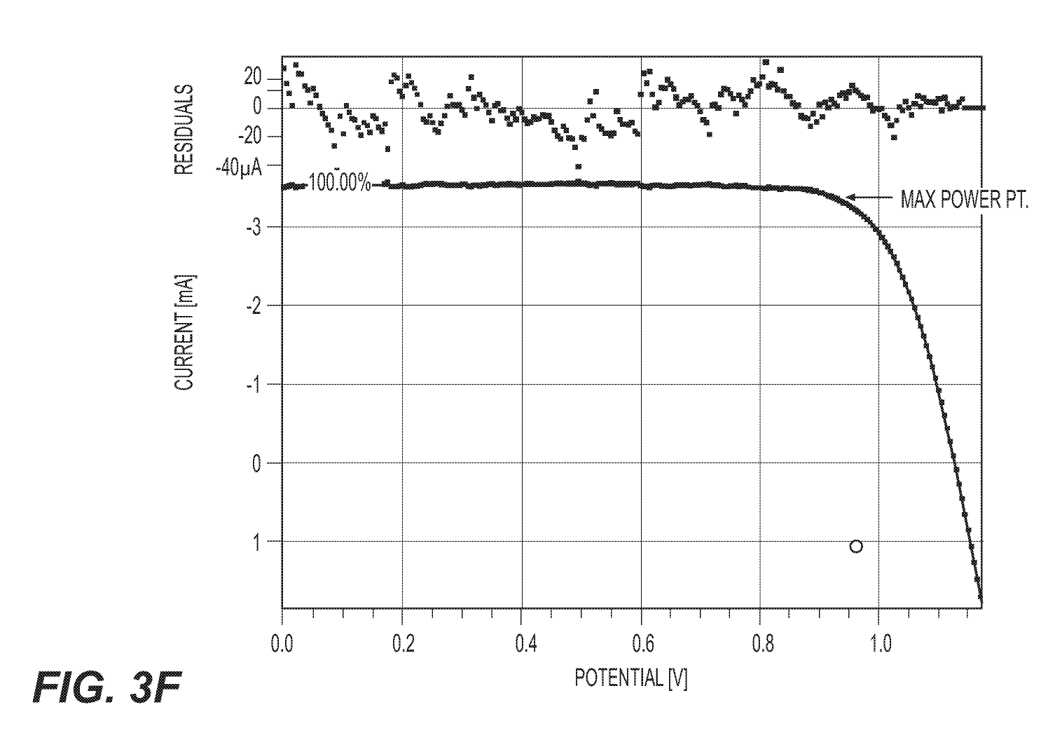

The solar cells were measured using a 450 W xenon light source (Oriel). The spectral mismatch between AM1.5G and the simulated illumination was reduced by the use of a Schott K113 Tempax filter (Prizisions Glas & Optik GmbH). The light intensity was calibrated with a Si photodiode equipped with an IR-cutoff filter (KG3, Schott), and it was recorded during each measurement. Current-voltage characteristics of the cells were obtained by applying an external voltage bias while measuring the current response with a digital source meter (Keithley 2400). The voltage scan rate was 10 mV s⁻¹ and no device preconditioning, such as light soaking or forward voltage bias applied for long time, was applied before starting the measurement. The starting voltage was determined as the potential at which the cell furnishes 1 mA in forward bias, and no equilibration time was used. The cells were masked with a black metal mask (0.16 cm²) to estimate the active area and reduce the influence of the scattered light. See FIGS. 3A-3F.

### Example 5

### Photovoltaic Characterization of Solar Cells

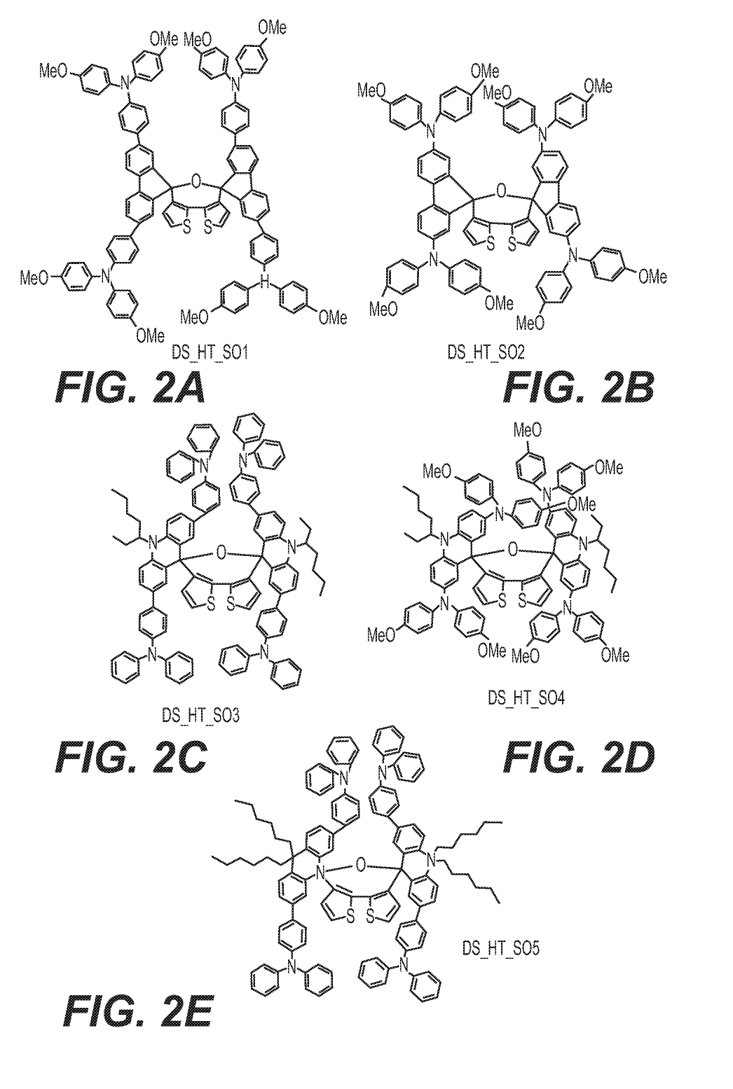

DS-HT-SO1, DS-HT-SO2, DS-HT-SO3, DS-HT-SO4, and DS-HS-SO5 are shown in FIGS. 2A, 2B, 2C, 2D, and 2E, respectively. DS-HT-SO1 is based on a spiro dithiophene structure and is not a dispiro-oxepine/dispiro-thiapine derivative for optoelectronic semiconductors, but is shown for the purpose of comparison. DS-HT-SO2, DS-HT-SO4, and DS-HT-SO6 are dispiro-oxepine/dispiro-thiapine derivatives for optoelectronic semiconductors.

Table 1 shows the photovoltaic properties of solar cells comprising a dispiro-oxepine/dispiro-thiapine derivative for optoelectronic semiconductors as hole transporting material and “mixed” perovskite (perovskite with mixed formamidinium methylammonium cations, and mixed iodine bromine anions) or standard perovskite (perovskite with methylammonium cation) in the sensitizer layer.

TABLE 1
Photovoltaic properties of solar cells

| | J_{sc} | V_{oc} | | PCE | |
|---|---|---|---|---|---|
| Compounds | [mAcm⁻²] | [mV] | FF | [%] | Perovskite |
| DS-HT-SO1 | 18.4 | 1069 | 0.33 | 6.6 | mixed |
| DS-HT-SO2 | 23.4 | 1091 | 0.78 | 18.5 | mixed |
| DS-HT-SO4 | 19.5 | 1118 | 0.649 | 14.3 | mixed |
| DS-HT-SO6 | 17.7 | 1119 | 0.734 | 14.6 | mixed |
| DS-HT-SO1 | 17.2 | 988 | 0.73 | 13.4 | standard |
| DS-HT-SO2 | 20.9 | 1100 | 0.76 | 17.2 | standard |
| DS-HT-SO4 | 18.8 | 1094 | 049 | 10.2 | standard |
| DS-HT-SO6 | 4.2 | 923 | 0.33 | 1.3 | standard |

In order to demonstrate the characteristics and properties of the dispiro-oxepine/dispiro-thiapine derivatives for optoelectronic semiconductors that make the compounds particularly suitable as a hole transporting material in semiconductors, the derivative DS-HT-SO2 (referred to in the following as DDOF) was test and compared to a conventional hole transporting material known as spiro-OMeTAD (2,2′,7,7′-tetrakis-(N,N′-di-4-methoxyphenylamine)-9,9′-spirobifluorene).

DDOF has the following structural formula:

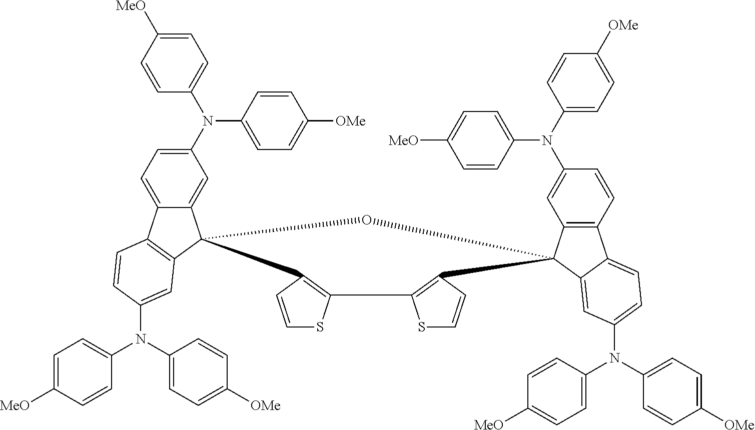

Typical perovskite-based solar cell (PSC) configuration is composed of an electron transporting material (ETM), infiltrated with the perovskite absorbing material and coated with a hole transporting material (HTM), which plays an important role to facilitate the holes from perovskite to the gold as a back contact. Spiro-based organic semiconductors have attracted considerable attention, more precisely, 2,2′,7,7′-tetrakis-(N,N′-di-4-methoxyphenylamine)-9,9′-spirobifluorene (spiro-OMeTAD) is selected as the benchmark HTM for PSC. However, the tedious multi-step synthesis of spiro-OMeTAD makes it prohibitively expensive and cost-ineffective. Moreover, high-purity sublimation-grade spiro-OMeTAD is required to obtain high-performance devices. So far, although a large number and different types of HTMs have been reported reaching efficiency of 16-19%, only very few candidates showed power conversion efficiency (PCE) values over 19%, mainly because of the additional interaction improving the hole transfer at the HTM/perovskite interface.

However, a PCE of 19.4% has been reached employing the present HTM 2,2′,7,7′-tetrakis-(N,N′-di-4-methoxyphenylamine)dispiro[fluorene-9,4′-dithieno[3,2-c:2′,3′-e]oxepine-6′,9″-fluorene], coded as DDOF, in PSC with improved stability, showing a potential as a low cost HTM to replace spiro-OMeTAD.

In the following examples, all reagents from commercial sources were used without further purification, unless otherwise noted. All reactions were performed under dry N₂ ambience, unless otherwise noted. All dry reactions were performed with glassware that was flamed under high-vacuum and backfilled with N₂. All extracts were dried over powdered MgSO₄ and solvents removed by rotary evaporation under reduced pressure. Flash chromatography was performed using Silicycle UltraPure SilicaFlash P60, 40-63 m (230-400 mesh). Thin-layer chromatography (TLC) was conducted with Merck KGaA pre-coated TLC Silica gel 60 F₂₅₄ aluminum sheets and visualized with UV.

¹H and ¹³C NMR spectra were recorded on a Bruker Avance-400 (400 MHz), Bruker AvanceIII-400 (400 MHz), Bruker DPX-400 (400 MHz) or Bruker DRX-600 (600 MHz) spectrometer and are reported in ppm using solvent as an internal standard: Chloroform-d at 7.24 ppm and 77.23 ppm for ¹H and ¹³C, respectively; THF-d8 at 1.72 and 3.58 ppm for ¹H, and 25.31, 67.21 for ¹³C, respectively. Data are reported as: s=singlet, d=doublet, t=triplet, q=quartet, p=pentet, m=multiplet, b=broad, ap=apparent; coupling constant(s) in Hz; integration.

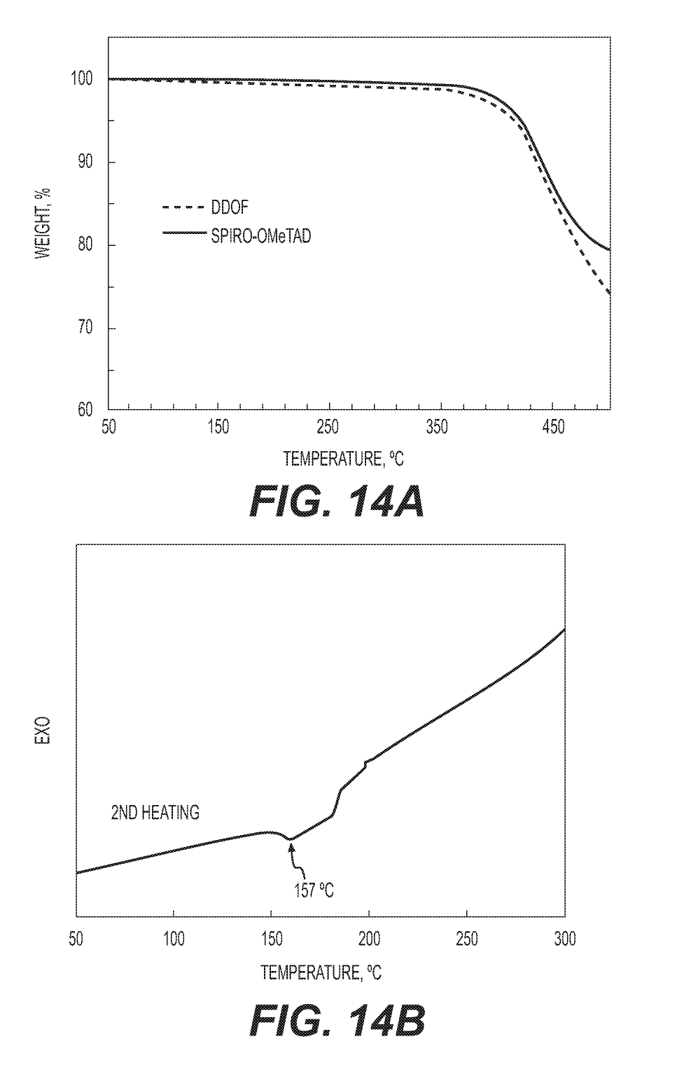

MS were recorded on Thermo Fisher Q Exactive HF Hybrid Quadrupole-Orbitrap Mass Spectrometer using matrix-assisted laser desorption/ionization (MALDI) technique (FIGS. 14A-B). UV-Vis spectra were measured with a Hewlett Packard 8453 UV-Vis spectrometer. Cyclic voltammetry (CV) was measured with an Autolab Eco Chemie cyclic voltammeter. Thermogravimetric analysis (TGA) data were collected using TGA 4000 from PerkinElmer (FIG. 18A). Differential scanning calorimetry (DSC) were recorded on DSC 8000 from PerkinElmer (FIG. 18B).

For conductivity measurements, solutions of spiro-OMeTAD and DDOF with molar concentrations of 30 mM in toluene were prepared and doped with 3 mol % FK-209. The solutions were spin-coated onto OFET substrates (Fraunhofer IPMS) at 4000 rpm for 30 s inside an inert atmosphere glovebox. The conductivity measurement was carried out at room temperature on a 2.5 am channel (by length) using an in-house developed probe station inside an Argon glovebox. The channel width was 10 mm and the channel height was 40 nm. The data were recorded using a potentiostat from Biologic by taking a hysteresis scan from −10 to 10 V at a scan rate of 2V s⁻¹. The conductivity was calculated from linear fit (Ohm's law).

Chemically etched FTO glass (Nippon Sheet Glass) was sequentially cleaned by sonication in a 2% Helmanex solution, acetone and ethanol for 30 min each, followed by a 15 min UV-ozone treatment. To form a 30 nm thick TiO₂ blocking layer, diluted titanium diisopropoxide bis(acetylacetonate) (TAA) solution (Sigma-Aldrich) in isopropanol was sprayed at 450° C. For the 200 nm mesoporous TiO₂ layer, mesoporous-TiO₂ layers were made by spin-coating a commercially available TiO₂ paste (Dyesol 30NRD). Substrates were baked at 500° C. for 30 min. Then, Li-doping of mesoporous TiO₂ is treated by spin coating a 0.03 M (0.1 M) solution of Li-TFSI in acetonitrile at 3000 rpm for 10 s, followed by another sintering at 500° C. for 20 min before the deposition of the perovskite layer. The perovskite absorber was (FAPbI₃)_{0.85}(MAPbBr₃)_{0.15}, prepared as follows. Mixed-perovskite precursor was prepared by mixing 1.15 m PbI₂, 1.10 m FAI, 0.2 m PbBr₂, 0.2 m MABr in a mixed solvent of DMF:DMSO=4:1 (volume ratio). Perovskite solutions are successively spin-coated in the glovebox as follows: first, 2000 rpm for 10 s with a ramp-up of 200 rpm s⁻¹; second, 6000 rpm for 30 s with a ramp-up of 2000 rpm s⁻¹. trifluorotoluene (110 ml) was dropped on the spinning substrate during the second spin-coating step 20 s before the end of the procedure, then films were annealed at 100° C. for 90 min. The hole-transporting materials were applied from a 20 mM solution in toluene. Tert-butylpyridine (Tbp), Tris(2-(1H-pyrazol-1-yl)-4-tert-butylpyridine)cobalt(III) (FK209) and Tris(bis(trifluoromethylsulfon-yl)imide) (Li-TFSI) were added as additives. Equimolar amounts of additives were added for hole-transporters: 330 mol % Tbp, 50 mol % Li-TFSI from a 1.8 M stock solution in acetonitrile and 3 mol % FK209 from a 0.25 M stock solution in acetonitrile. The final HTM solutions were spin-coated onto the perovskite layers at 4000 rpm for 30 s. The gold electrode of 80 nm was deposited by thermal evaporation in high vacuum conditions.

The solar cells were measured using a 450 W xenon light source (Oriel). The spectral mismatch between AM1.5G and the simulated illumination was reduced by the use of a Schott K113 Tempax filter (Prazisions Glas & Optik GmbH). The light intensity was calibrated with a Si photodiode equipped with an IR-cutoff filter (KG3, Schott), and it was recorded during each measurement. Current-voltage characteristics of the cells were obtained by applying an external voltage bias while measuring the current response with a digital source meter (Keithley 2400). The voltage scan rate was 10 mV s⁻¹ and no device preconditioning, such as light soaking or forward voltage bias applied for long time, was applied before starting the measurement. The starting voltage was determined as the potential at which the cells furnishes 1 mA in forward bias, no equilibration time was used. The cells were masked with a black metal mask (0.16 cm²) to fix the active area and reduce the influence of the scattered light.

### Example 6

### Synthesis of DDOF

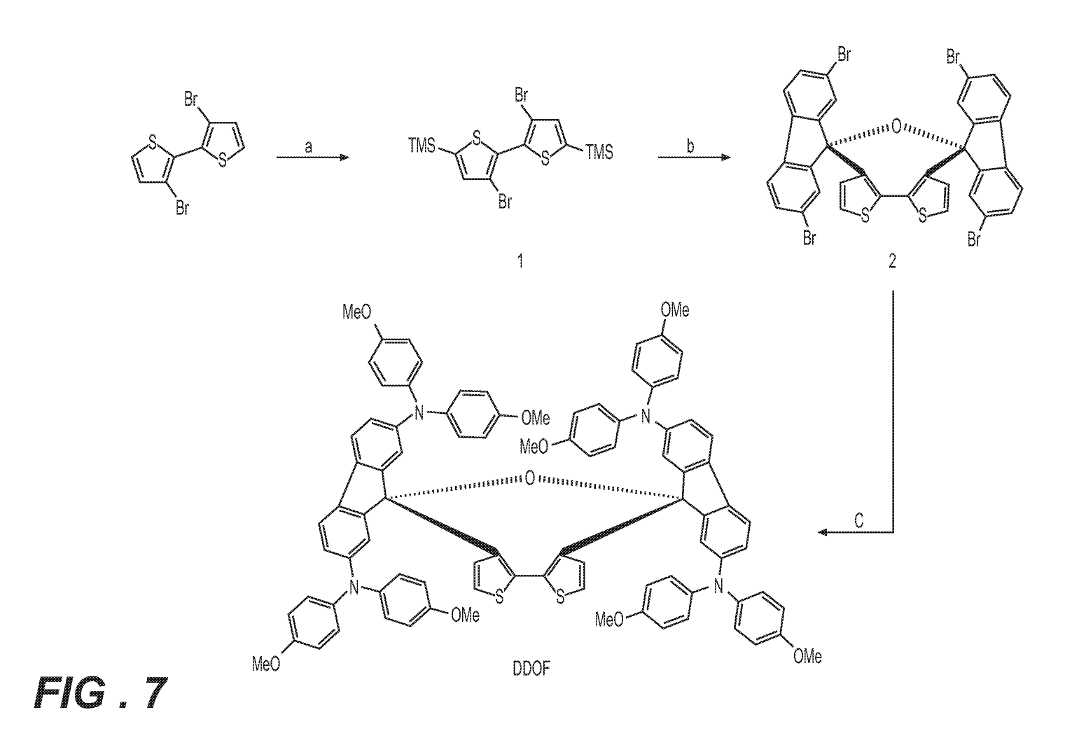

The general synthesis procedure for the preparation of DDOF is shown in FIG. 7. Firstly, commercially available precursor 3,3′-dibromo-2,2′-bithiophene has been protected with trimethylsilyl groups, obtaining 1. By adding a stoichiometric amount of n-BuLi, 5,5′-ditrimethylsilyl-3,3′-dilithio-2,2′-bithiophene was generated in situ, followed by treatment with an excess of 2,7-dibromofluorenone to form the intermediate carbinol. Condensing in acetic acid in the presence of a catalytic amount of hydrochloric acid, two hydroxyls are involved in intramolecular H-bonding that facilitated an etherification reaction between these hydroxyl groups with closing of the seven membered ring. Finally, dispiro-oxepine derivative 2 have been equipped with 4,4′-dimethoxydiphenylamine units via palladium-catalyzed Buchwald-Hartwig C≡N cross coupling reaction to result in the final HTM DDOF having original 3D nodes.

In detail, the 5,5′-Bis(trimethylsilyl)-3,3′-dibromo-2,2′-bithiophene 1 intermediate in the foregoing synthesis was prepared as follows. To a solution of commercially available 3,3′-dibromo-2,2′-bithiophene (5 g, 15.4 mmol, 1 equiv) in dry THF (50 mL), lithium diisopropylamide, 2.0 M (17 mL, 34 mmol, 2.2 equiv) was added dropwise at −78° C. After stirring for 1 h, trimethylsilyl chloride (4.3 mL, 34 mmol, 2.2 equiv) was added dropwise at −78° C. The reaction mixture was then allowed to warm to room temperature and stirred overnight. Water was added to quench the reaction. The resulting mixture was extracted with hexane. This crude residue was purified by flash chromatography with pure hexane. Yield: (6.5 g, 90%). Colorless needles. ¹H NMR (400 MHz, CDCl₃) δ 7.18 (s, 2H), 0.37 (s, 18H). 13C NMR (100 MHz, CDCl₃) δ 143.81, 137.44, 134.24, 113.37, −0.36. C₁₄H₂₀Br₂S₂Si₂ [M⁺] exact mass=465.8912 MS (MALDI-TOF)=465.880.

The tetrabromo intermediate, namely, 2,2″,7,7″-tetrabromodispiro[fluorene-9,4′-dithieno[3,2-c:2′,3′-e]oxepine-6′,9″-fluorene] 2, was prepared as follows. To a solution of 1 (2 g, 4.3 mmol, 1 equiv) in 50 mL of dry THF at −78° C. under argon atmosphere, n-BuLi (2.5 M in hexanes, 3.6 mL, 9 mmol, 2.1 equiv) is dropwise added. After 2 h at the same temperature, 2,7-dibromofluorenone (3.65 g, 10.8 mmol, 2.5 equiv) in THF (50 mL) is added to the mixture dropwise, and the solution was warmed to room temperature and stirred overnight. The mixture was washed with water, extracted with DCM, and the combined organic phases are dried over MgSO₄. The solvent is evaporated and the crude product is precipitated in ethanol to afford a white solid and is used in the next step without further purification. Obtained solid was dissolved in boiling acetic acid (100 mL), and 1 mL of concentrated hydrochloric acid was added. After refluxing for 2 h, the mixture was washed with water and extracted with DCM, and the combined organic phases dried over MgSO₄. This crude residue was purified by flash chromatography with pure 20% DCM in hexane. (1.4 g, 41%). ¹H NMR (400 MHz, CDCl₃) δ 7.49 (dd, J=7.9 Hz, 4H), 7.44 (d, J=8.1 Hz, 4H), 7.18 (d, J=5.1 Hz, 2H), 7.09 (d, J=1.5 Hz, 4H), 6.39 (d, J=5.1 Hz, 2H). 13C NMR (100 MHz, CDCl₃) δ 151.1, 140.8, 137.5, 134.4, 132.4, 129.1, 128.9, 124.8, 122.2, 121.4, 88.1. C₃₄H₁₆Br₄OS₂ [M⁺] exact mass=819,7376 MS (MALDI-TOF)=819.721.

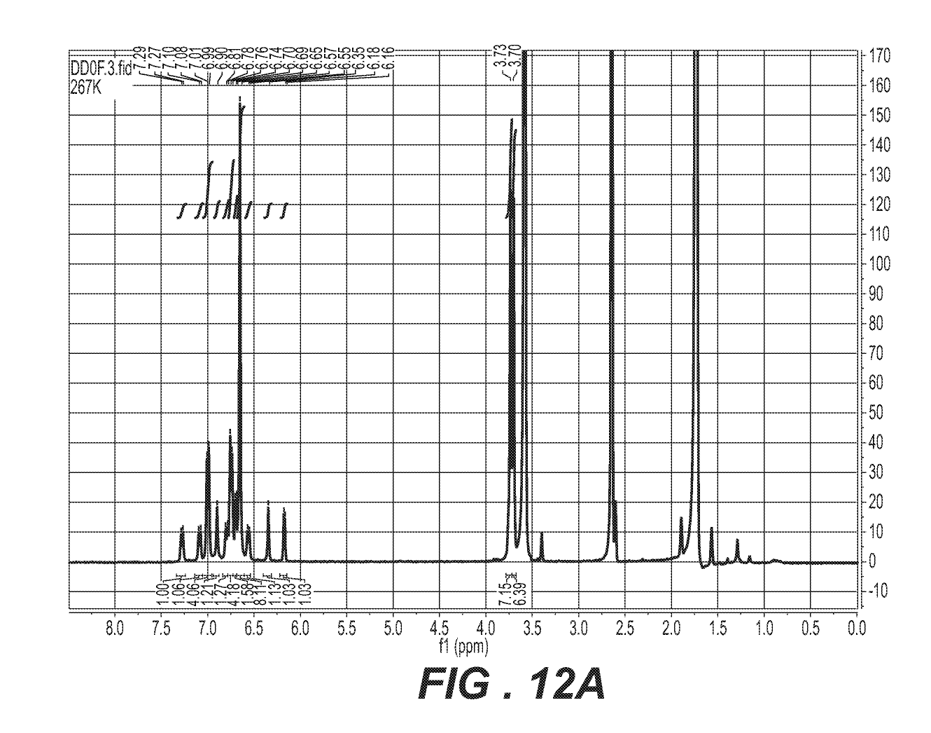

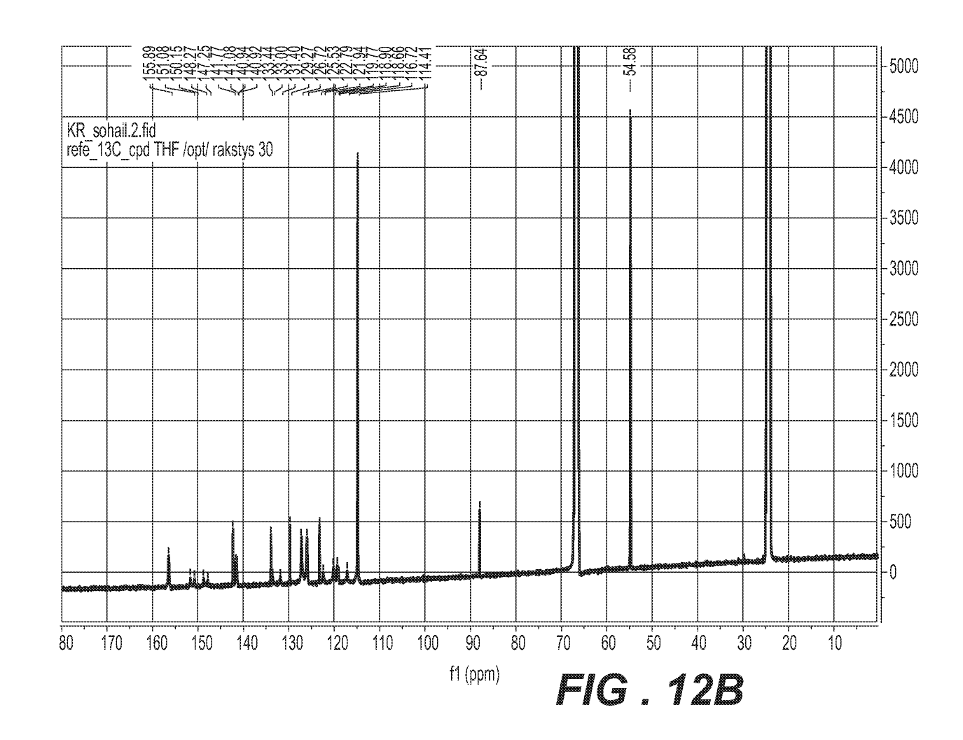

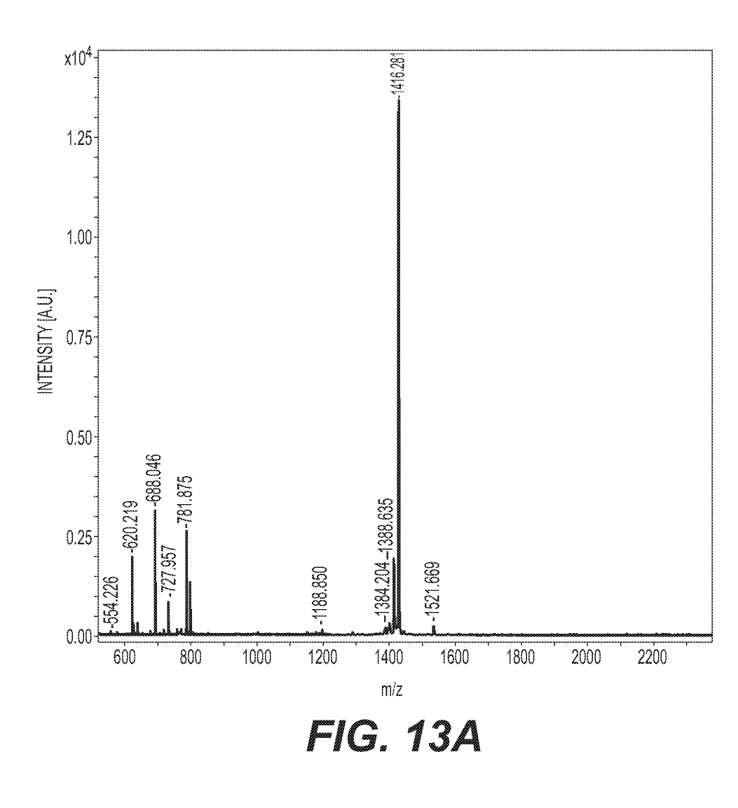

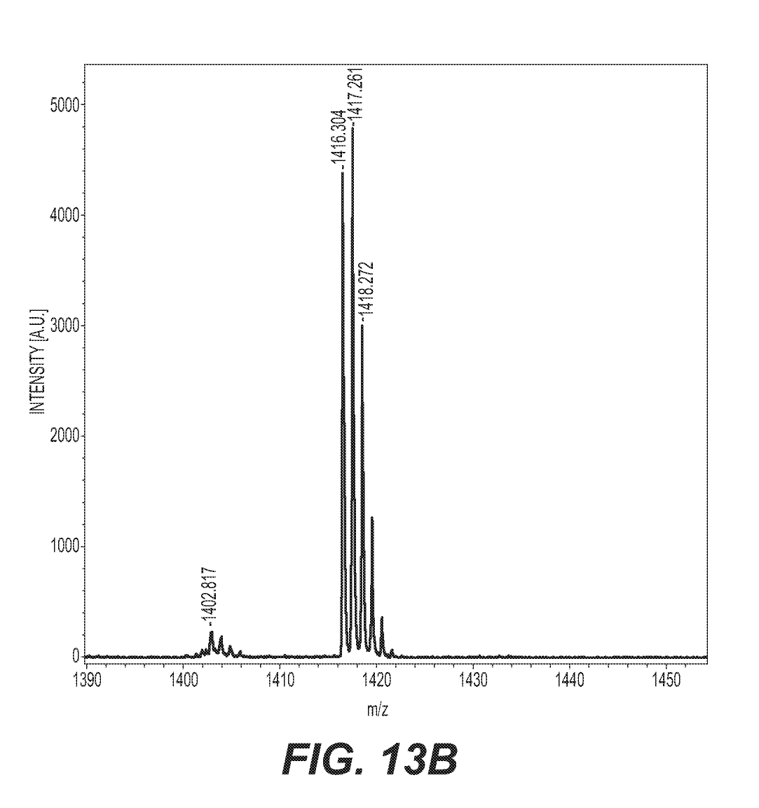

The last step to obtain the final product, 2,2′,7,7′-tetrakis-(N,N′-di-4-methoxyphenylamine)dispiro[fluorene-9,4′-dithieno[3,2-c:2′,3′-e]oxepine-6′,9″-fluorene](DDOF), was performed as follows. In a 50 mL Schlenk-tube, 400 mg of 2 (0.49 mmol, 1 equiv), 560 mg commercially available 4,4′-dimethoxydiphenylamine (2.5 mmol, 5 equiv), and 280 mg t-BuONa (2.9 mmol, 6 equiv) were dissolved in 20 mL dry toluene and degassed for 20 min with N₂. After the addition of 70 mg Pd₂dba₃ (0.075 mmol, 15%) and 70 mg Xphos (0.15 mmol, 30%), the reaction was refluxed overnight. The reaction was then diluted with DCM and flashed through a plug of MgSO₄ to remove inorganic salts and metallic palladium. This crude residue was purified by flash chromatography with 30% THF in hexane. Isolated compound was dissolved in THF and dropped into MeOH, precipitate was collected by filtration, washed with MeOH and dried. 420 mg (60% yield) of pale yellow solid was obtained. ¹H NMR (400 MHz, THF, 267K) δ 7.28 (d, J=8.0 Hz, 2H), 7.09 (d, J=8.1 Hz, 2H), 7.00 (d, J=7.9 Hz, 8H), 6.90 (s, 2H), 6.80 (d, J=8.4 Hz, 2H), 6.75 (d, J=7.8 Hz, 8H), 6.69 (d, J=5.2 Hz, 2H), 6.65 (s, 16H), 6.56 (d, J=7.8 Hz, 2H), 6.35 (s, 2H), 6.17 (d, J=5.3 Hz, 2H), 3.73 (s, 12H), 3.70 (s, 12H). ¹³C NMR (100 MHz, THF) δ 155.89, 151.08, 150.15, 148.27, 147.25, 141.77, 141.08, 140.94, 140.92, 133.44, 133.00, 131.40, 129.27, 126.72, 125.53, 122.79, 121.94, 119.77, 118.90, 118.66, 116.72, 114.41, 87.64, 54.58. C₉₀H₇₂N₄O₉S₂[M⁺] exact mass=1416,4741 MS (MALDI-TOF)=1416.304. The ¹H NMR and the ¹³C NMR are shown in FIGS. 12A and 12B, respectively. The MS spectra are shown in FIGS. 13A and 13B. Thermographic analysis of DDOF is shown in FIG. 14A and differential scanning calorimetry is shown in FIG. 14B.

### Example 7

### Crystallographic Analysis of DDOF

A crystallographic analysis was performed as follows. Intensity data were collected on an Rigaku Supernova, second edition, 2015 diffractometer by using graphite-monochromatized Cu Ka radiation (λ=1.5418 Å) at room temperature [293 K]. A summary of the crystallographic data, the data collection parameters, and the refinement parameters are given in Table 2. CCDC-1495258 contains the supplementary crystallographic data, and the data can be obtained free of charge from The Cambridge Crystallographic Data Centre.

TABLE 2
Crystallographic Parameters of DDOF and spiro-OMeTAD

| Parameter | DDOF | Spiro-OMeTAD |
|---|---|---|
| Empirical formula | C₉₀H₇₂N₄O₉S₂ | C₈₁H₆₈N₄O₈ |
| Formula weight | 1417.71 | 1225.455 |
| Crystal color, habit | Colorless, needle | Colorless, needle |
| Crystal system | monoclinic | triclinic |
| a, Å | 25.4756(19) | 13.1111(7) |
| b, Å | 16.5543(11) | 16.1465(7) |
| c, Å | 19.5340(13) | 16.9214(9) |
| α, deg | 90 | 75.200(4) |
| β, deg | 107.428(8) | 85.670(4) |
| γ, deg | 90 | 75.891(4) |
| ν, Å³ | 7859.91(1) | 3358.57 |
| ρ calc, g/cm³ | 1.19792 | 1.267 |
| Space group | P 1 21/c 1 (14) | P −1 |
| Z value | 4 | 2 |
| Temperature, K | 293 | 140 |
| no. of reflections measured | 16089 | 23601 |
| no. of variables | 1090 | 909 |
| Residuals: R; wR2 | 0.0891, 0.2758 | 0.0419, 0.1068 |

The data reduction was carried out by Crysalis PRO (1). The solution and refinement were performed by SHELX (2). The crystal structure was refined using full-matrix least-squares based on F2 with all non hydrogen atoms anisotropically defined. Hydrogen atoms were placed in calculated positions by means of the “riding” model.

### Example 8

### Geometric and Electronic Properties of DDOF

The geometrical and electronic properties of the compound were performed with the Gaussian 09 program package. The calculation was optimized by means of the B3LYP (Becke three parameters hybrid functional with Lee-Yang-Perdew correlation functional) with the B3LYP/6-31G* atomic basis set. The excitation transitions of DDOF were calculated using time-dependent density functional theory (TD-DFT) calculations with B3LYP/6-31g*. Molecular orbitals were visualized using Gaussview.

In an attempt to gain more insight into electronic structure of compound DDOF, MO calculations were performed at the TD-DFT and DFT levels of theory, respectively, using the B3LYP functional and 6-31G(d) basis set employing the Gaussian 09 package. As such, geometry optimizations for the ground state were performed by density functional theory (DFT) at the B3LYP/6-31G* level, the frontier molecular orbitals of the molecules for the target molecule were calculated with an isovalue of 0.025 obtained by TD-DFT at the B3LYP/6-31G* level, and all the DFT calculations were carried out with the Gaussian 09 package 2. The optimized geometry of DDOF was calculated. The optimized molecular geometry shows that four diphenyl amine side arms are extended from the two coplanar fluorene groups forming four redox active centers. The two fluorene planes are roughly orthogonal to each other with an angle of 89.66°. Due to the non-aromatic nature of the central seven-member ring, the planes of two thiophene rings are twisted with a dihedral angle of 26.36°.

The electron density distributions in the highest occupied molecular orbital (HOMO) and the lowest unoccupied molecular orbital (LUMO) were calculated. The HOMO and HOMO-1 of DDOF are close in energy and spread over the one of the two bis(dimethoxyphenyl amine) fluorene groups respectively, resulting from the antibonding interaction of all the aromatic rings. Due to the fact that the central seven-member ring breaks the conjugation among the three main conjugated moieties, the interaction between their virtual orbitals is less pronounced. The LUMO of DDOF is a π* orbital confined on the bithiophene groups, and the LUMO+1 is a π* orbital that is mainly delocalized over the two fluorine groups.

### Example 9

### UV-VIS and Cyclic Voltammetry of DDOF

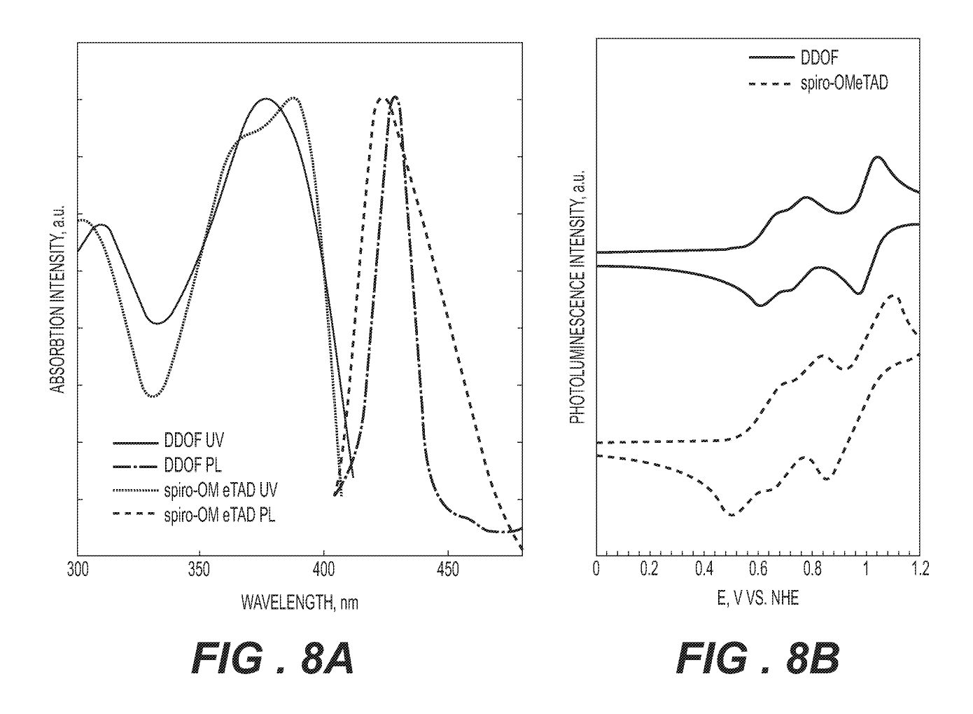

The normalized UV-vis absorption and photoluminescence spectra of DDOF and spiro-OMeTAD in THF are shown in FIG. 8A. Both exhibit an almost identical absorption band in the UV region, with the absorption maximum around 385 nm. Comparing with spiro-OMeTAD, DDOF shows a narrow emission band centered at 426 nm. Optical band gap (E_{g}) determined from the onset of absorption were 3.02 eV for DDOF, which is almost identical to that of spiro-OMeTAD. To compare the energy levels of the new HTM with spiro-OMeTAD, we performed cyclic voltammetry (CV) measurement. The data derived from the ground-state oxidation potential (E^{HOMO}) estimated from cyclic voltammogram, shown in FIG. 8B, are summarized in Table 3.

TABLE 3
Optical and electrochemical properties comparison

| | λ_{abs}, | λₑₘ, | E_{g}, | E^{HOMO} vs. | HOMO, | LUMO, |
|---|---|---|---|---|---|---|
| ID | nm^{[a]} | nm^{[a]} | eV^{[b]} | NHE, V^{[c]} | eV^{[c]} | eV^{[d]} |
| DDOF | 378 | 426 | 3.02 | 0.63 | −5.07 | −2.05 |
| spiro- | 390 | 424 | 3.05 | 0.60 | −5.04 | −1.99 |
| OMeTAD | | | | | | |

^{[a]}Absorption was photoluminescence measured in THF solution.
^{[b]}Determined from the UV-vis absorption onset.
^{[c]}Measured in DCM/tetra-n-butylammonium hexafluorophosphate (0.1M) solution, using glassy carbon working electrode, Pt reference electrode, and Pt counter electrode with Fc/Fc⁺ as an internal standard. Potentials were converted to the normal hydrogen electrode by addition of +0.624 V and −4.44 eV to the vacuum, respectively.
^{[d]}Calculated from LUMO = HOMO + E_{g}.

The HOMO value of DDOF was estimated to be −5.07 eV vs. vacuum, which is slightly destabilized compared with that of spiro-OMeTAD (−5.04 eV). Considering the valence band of double-mixed perovskite is at −5.65 eV (vs. vacuum), DDOF shows enough over potential leading to efficient photogenerated charge transfer at the interface.

### Example 10

### Sample Perovskite Solar Cells

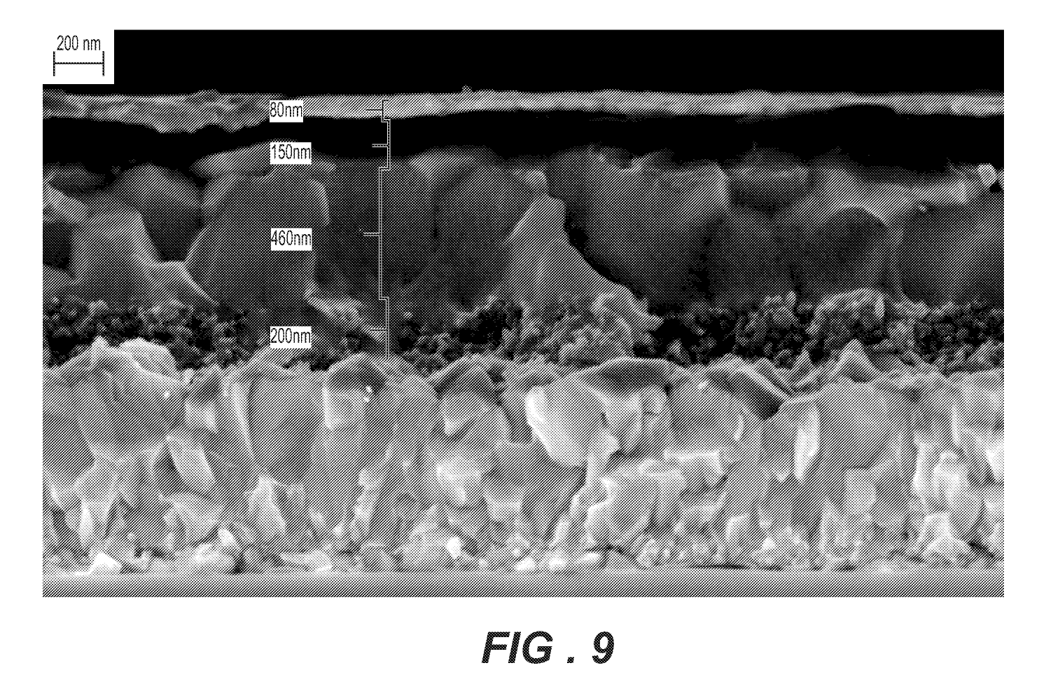

To demonstrate the ability of DDOF act as HTM, PSCs were prepared with (FAPbI₃)_{0.85}(MAPbBr₃)_{0.15} perovskite as the absorber. FIG. 9 displays the cross-section images of the PSCs analyzed by field-emission scanning electron microscope (SEM). The device is made by ˜460 nm perovskite atop 200 nm thick mesoporous TiO₂ layer, which was deposited on FTO glass coated with ˜50 nm compact TiO₂. The device is completed by ˜150 nm thick HTM and 80 nm gold as back contact.

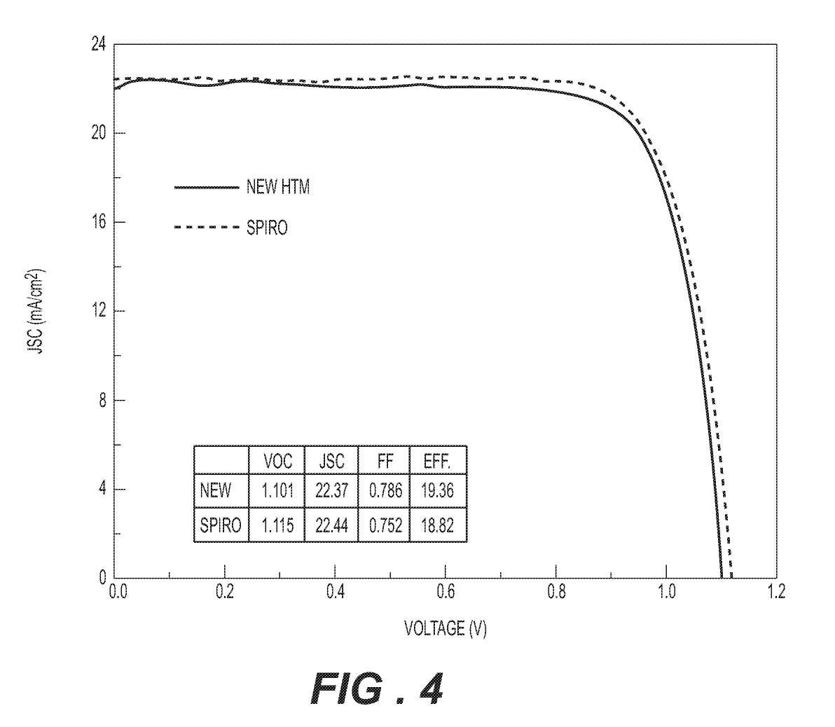

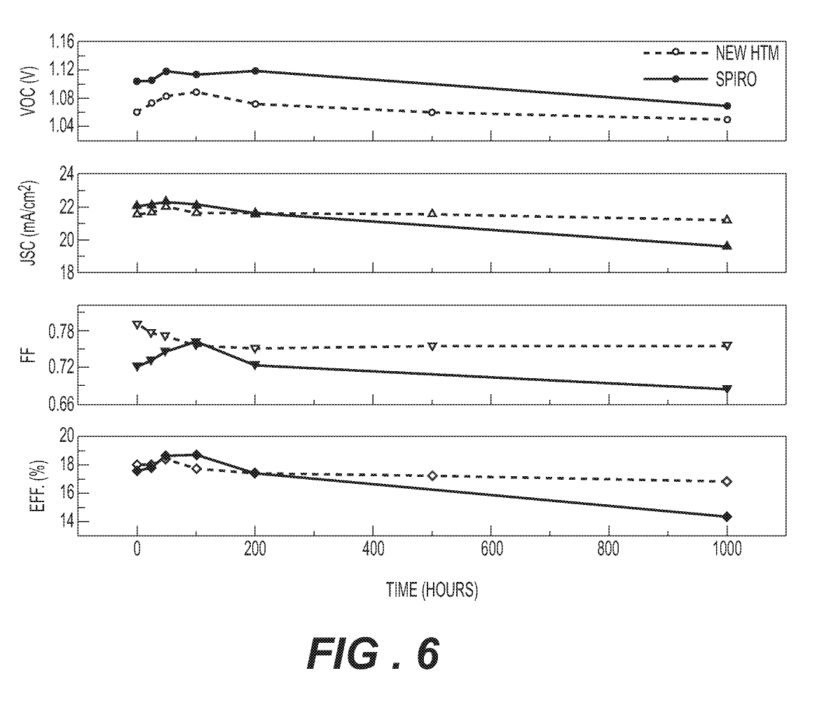

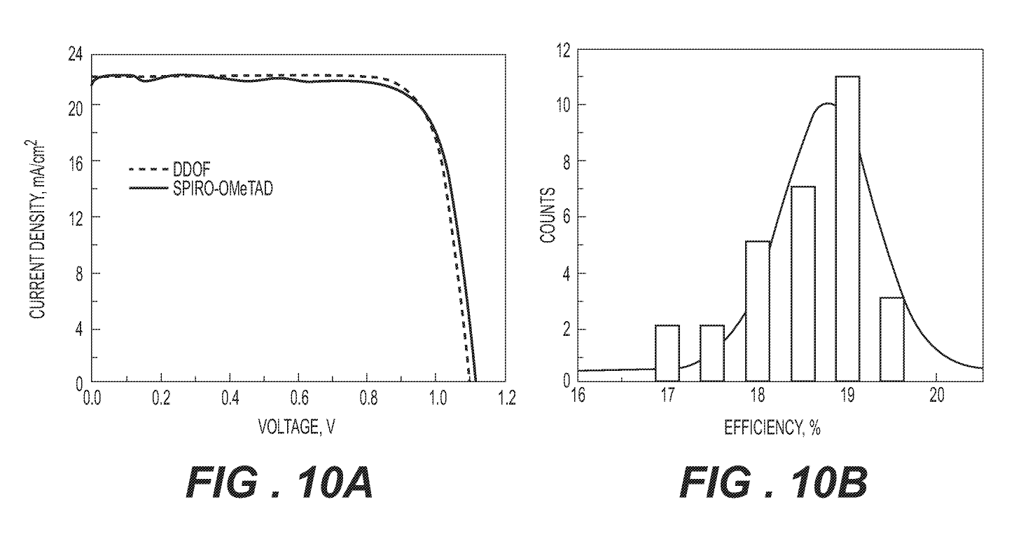

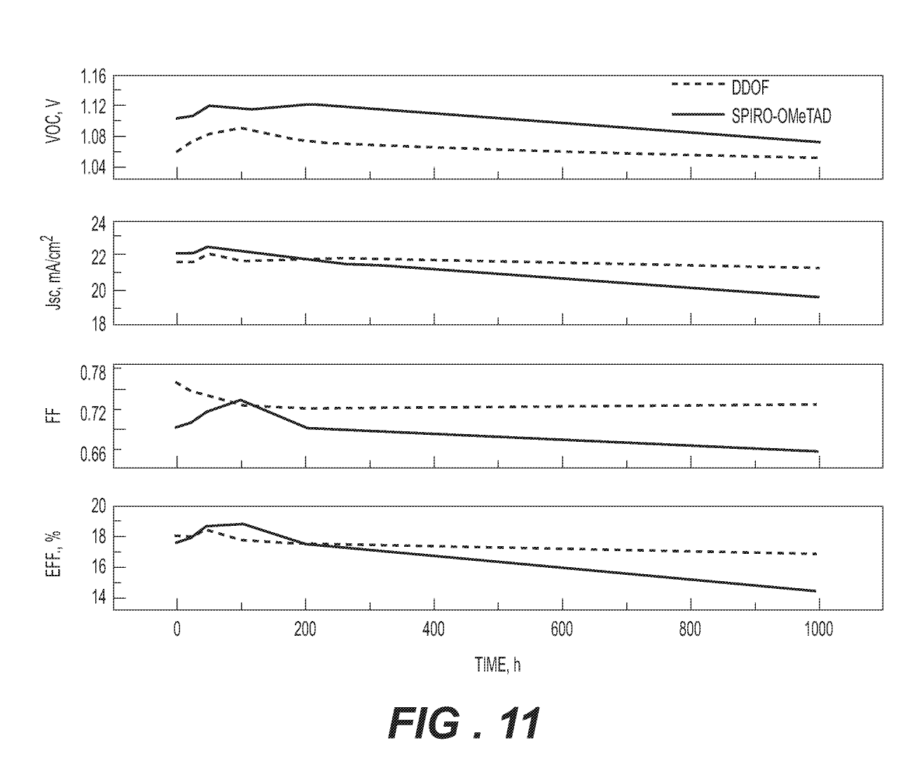

FIG. 4 and FIG. 10A illustrate the current-voltage (J-V) traces collected under simulated solar illumination (AM 1.5, 100 mW cm²) for the best DDOF PSC among 30 devices compared to spiro-OMeTAD. Statistical distribution of the PCE of the 30 DDOF devices is shown in FIG. 10B. Summarized electrical output characteristics are reported in Table 4. The device with DDOF as HTM possesses an open-circuit voltage (V_{OC}) of 1101 mV, a short circuit current density (J_{SC}) of 22.37 mA cm⁻², and a fill factor (FF) of 0.79, yielding a PCE of 19.4%. For comparison, the reference device based on spiro-OMeTAD achieved an overall PCE of 18.8%, which is mainly because of lower FF, despite slightly higher J_{SC} and V_{OC}. FIGS. 6 and 11 show the V_{OC}, J_{SC}, FF, and PCE curves.

TABLE 4
Solar cell performance parameters, extracted from J-V curves

| | Jsc, | Voc, | | PCE |
|---|---|---|---|---|
| ID | mA/cm² | mV | FF | (%) |
| DDOF | 22.37 | 1101 | 0.79 | 19.4 |
| spiro-OMeTAD | 22.44 | 1115 | 0.75 | 18.8 |

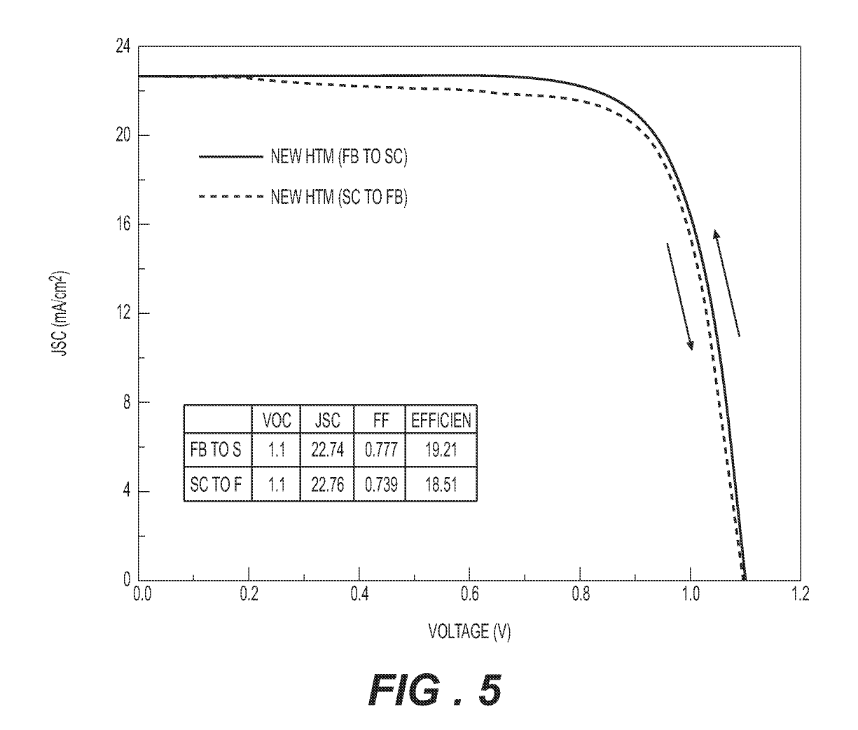

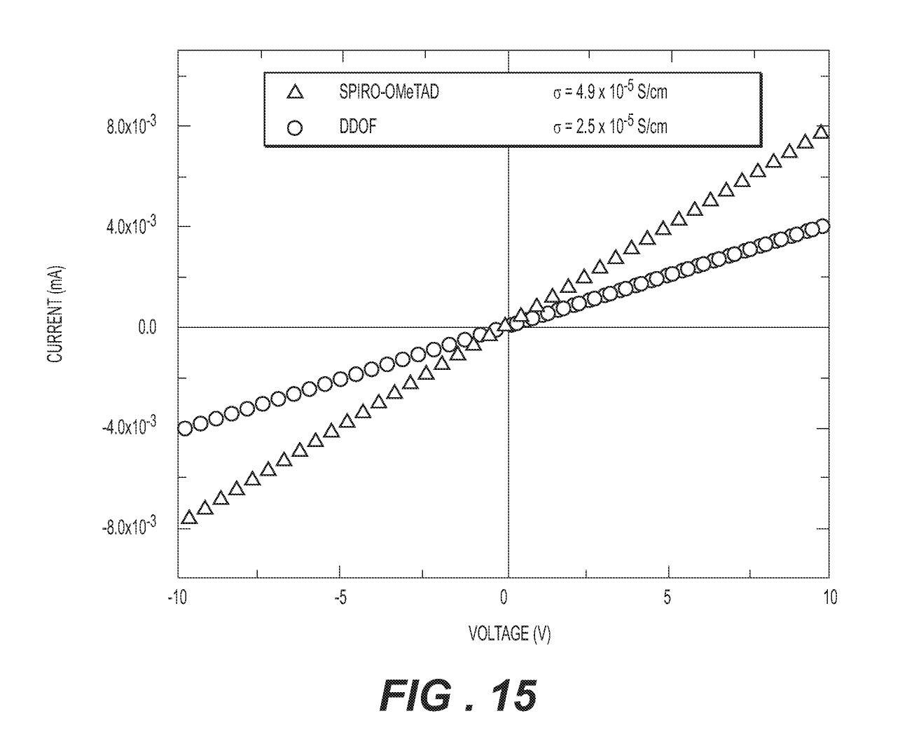

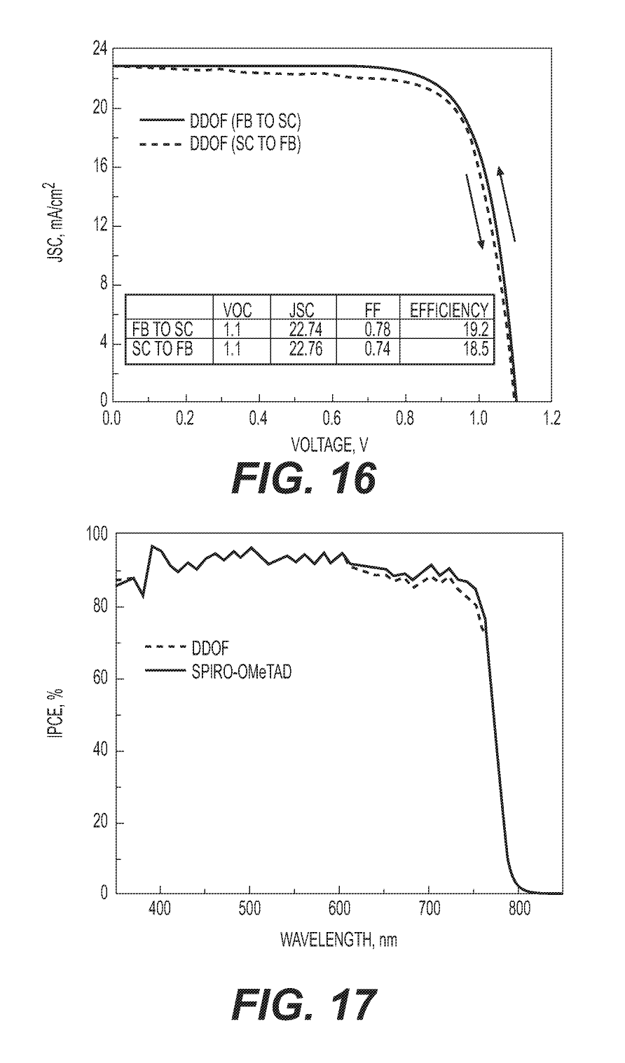

Further, FIGS. 5 and 16 illustrate the current (J)-voltage (V) curves of the solar cell with DDOF collected under AM1.5 simulated sun light. The curves were recorded scanning at 0.01 V s⁻¹ from forward bias (FB) to short circuit condition (SC) and the other way round. FIG. 15 shows conductivity measurements of the HTMs on OFET substrates.

The incident photon-to-current efficiency (IPCE) (shown in FIG. 17) of the perovskite devices as a function of wavelength shows that the device with DDOF as the HTM exhibits IPCE above 90% from 400 nm covering all the visible region to 700 nm.

The stability of PSCs is a key factor that plays a major role in their commercialization potential. FIG. 11 presents the stability of the devices containing DDOF and spiro-OMeTAD as HTMs. Stability tests were carried out keeping devices in the dark at a relative humidity of 10% without any encapsulation for 1000 hours. The cells with DDOF maintained around 95% of their initial PCE, while the PCE of devices with spiro-OMeTAD dropped by 20% under identical conditions. The general tendency among 10 devices indicates better long-term stability of our developed HTM.

It is to be understood that the dispiro-oxepine/dispiro-thiapine derivatives for optoelectronic semiconductors are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed dispiro-oxepine/dispiro-thiapine derivatives for optoelectronic semiconductors.

We claim:

1. A dispiro-oxepine derivative for optoelectronic semiconductors comprising a compound of formula (Ia) or (Ib): 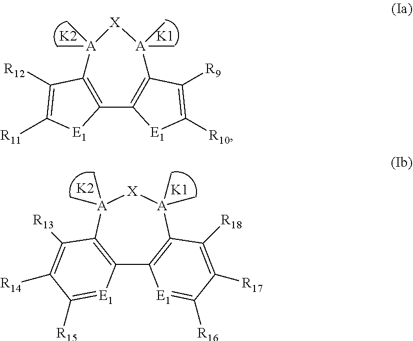
combined with two moieties selected from K1 and K2: 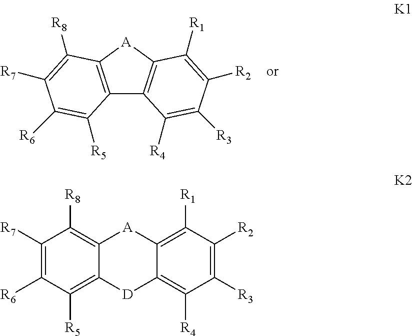
wherein
A is independently selected from Si or C;
D is a heteroatom independently selected from O, S, and N and can be bare or substituted by a H, alkyl, or aryl chain to form D-H, D-Alkyl, or D-Aryl, respectively;
E₁ and E₂ are independently selected from O, S, and N;
X is independently selected from O, S, and Se;
at least one R₁, R₂, R₃, R₄, R₅, R₆, R₇, and R₈ is independently selected from substituents comprising 1-50 carbons, 1-20 heteroatoms being selected from O, S, N, and 0-2 P-hydrocarbyl, the substituents being further substituted by further substituents selected from H, halogen, C1-C10 alkyl, C1-C10 alkoxy group, C1-C10 alkylthio (—S-alkyl) and —C≡N; wherein any one of R¹, R², R³, R⁴, R⁵, R⁶, R⁷, and R⁸ different from said substituents comprising 1-50 carbons, 1-20 heteroatoms and 0-2 P-hydrocarbyl is H; and
R₉, R₁₀, R₁₁, R₁₂, R₁₃, R₁₄, R₁₅, R₁₆, R₁₇, and R₁₈ are independently selected from H; halogen selected from Cl, F, Br, or I; C1-C30 alkyl; C1-C30 heteroalkyl; C4-C20 aryl; C4-C20 heteroaryl; C4-C30 alkylaryl group; C4-C30 aryloxy group; or C4-C20 heteroaryloxy group; wherein the heteroatom is independently selected from O, S, N, —P(═O)—, and —C≡N; wherein if the alkyl, heteroalkyl, or alkylaryl comprise 3 or more carbons, the alkyl, heteroalkyl, or alkylaryl may be linear, branched, or cyclic; and from a substituent as defined above for R₁, R₂, R₃, R₄, R₅, R₆, R₇, and R₈ of the substituent R₉, R₁₀, R₁₁, R₁₂, R₁₃, R₁₄, R₁₅, R₁₆, R₁₇, and R₁₈ in K1 and/or K2.

2. The dispiro-oxepine derivative according to claim 1, wherein the compound composed from formula (Ia) or (Ib) and two moieties selected from K1 and K2 comprises a compound of formula (IIa), (IIb), (IIIa), or (IIIb),

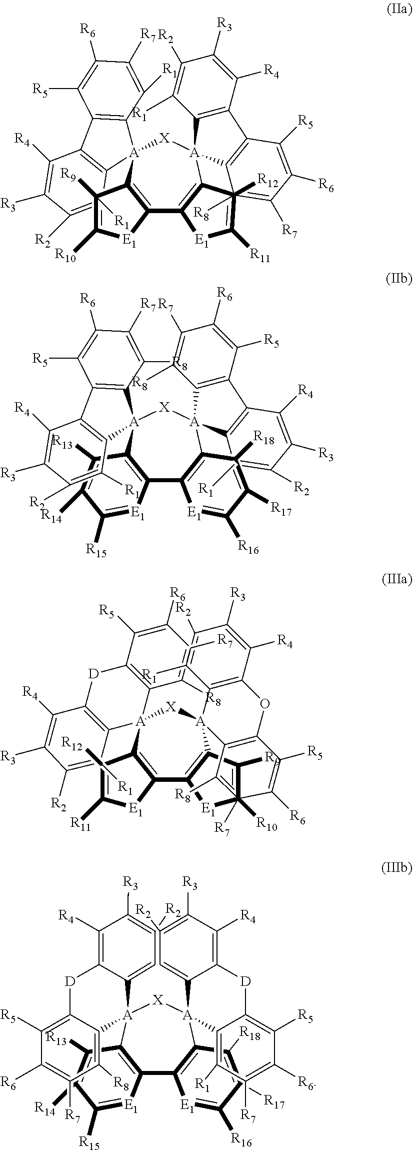

3. The dispiro-oxepine derivative according to claim 1, wherein at least one of R₁, R₂, R₃, R₄, R₅, R₆, R₇, and R₈ is different from H and is independently selected from an amino group, P-hydrocarbyl, or a mono- or polycyclic system comprising fused aromatic rings or monocyclic aromatic rings bound together by covalent bond, a ring comprising 0, 1, or 2 heteroatoms selected from O, S, and N;
wherein the amino group, P-hydrocarbyl, and mono- or polycyclic system may be further substituted by H, halogen, R₁, —NR₁R₂, —O—R₁, —P(═O) R₁R₂, or —S—R₁;
wherein R₁ and R₂ are independently selected from C4-C20 aryl, C4-C20 heteroaryl, C4-C20 aryloxy group, C4-C20 heteroaryloxy group, C4-C20 alkoxyaryl, C4-C20 alkoxyheteroaryl, C4-C20 aryl aryloxy group, C4-C20 heteroaryl aryloxy group, C1-C20 alkyl, C1-C20 alkoxy group, C1-C20 alkoxyalkyl, C1-C20 alkylthio, C2-C20 alkenyl, and C2-C20 alkynyl; and
wherein the alkyl, alkoxy, alkoxyalkyl, alkenyl, and alkynyl, if comprising 3 or more carbons, may be linear, branched, or cyclic.

4. The dispiro-oxepine derivative according to claim 1, wherein at least one of R₁, R₂, R₃, R₄, R₅, R₆, R₇, and R₈ is different from H and is independently selected from a substituent of formula (1) 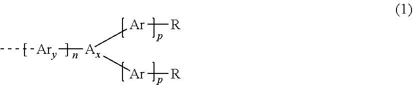
wherein
n is an integer selected from 0, 1, or 2;
p is an integer selected from 0, 1, or 2;
A_{X} is independently selected from N or P(═O);
Ar_{y} and Ar are independently selected from a monocyclic system or a polycyclic system comprising fused aromatic rings or conjugated monocyclic aromatic rings, the rings comprising 0, 1, or 2 heteroatoms selected from O, S and N, and further substituted in addition to R by other substituents independently selected from H, halogen, C1-C10 alkyl, C1-C10 alkoxy group, C1-C10 alkylthio (—S-alkyl), and —C≡N; and
R is independently selected from H, R₁, —NR₁R₂, —O—R₁, —P(═O) R₁R₂, —S—R₁, or halogen, wherein R₁ and R₂ are independently selected from C4-C20 aryl, C4-C20 heteroaryl, C4-C20 aryloxy group, C4-C20 heteroaryloxy group, C4-C20 alkoxyaryl, C4-C20 alkoxyheteroaryl, C4-C20 aryl aryloxy group, C4-C20 heteroaryl aryloxy group, C1-C20 alkyl, C1-C20 alkoxy group, C1-C20 alkoxyalkyl, C1-C20 alkylthio, C2-C20 alkenyl and C2-C20 alkynyl, wherein the alkyl, alkoxy, alkoxyalkyl, alkenyl and alkynyl, if comprising 3 or more carbons, may be linear, branched, or cyclic, and wherein aryl, heteroaryl, alkyl, alkenyl, and alkynyl may be further substituted by alkoxy group, alkylthio group, and alkyl.

5. The dispiro-oxepine derivative according to claim 4, wherein Ar_{y} and Ar are independently selected from moieties according to any one of formula (2) to (19) 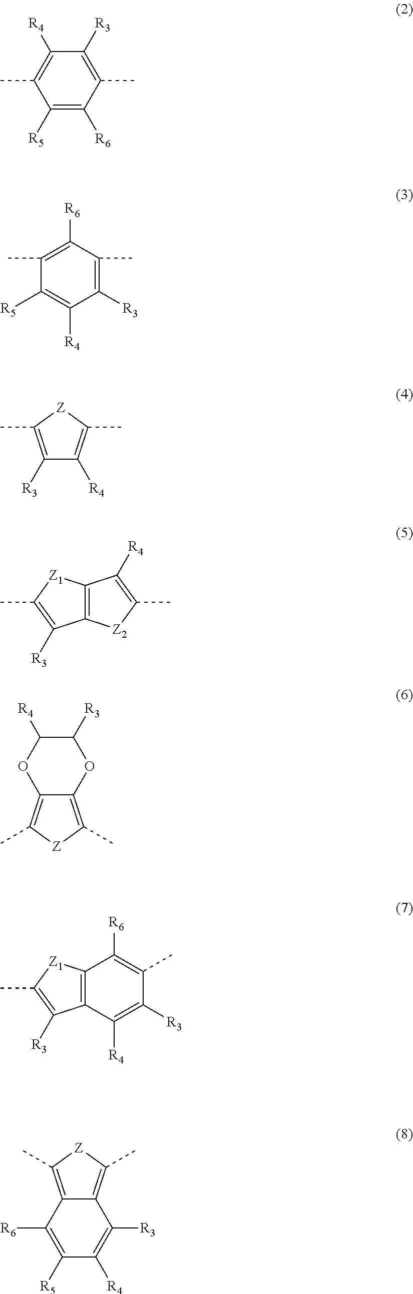 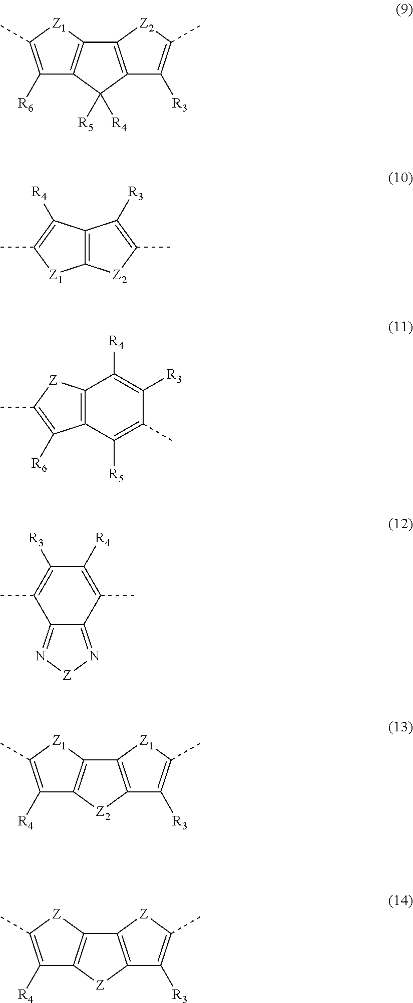 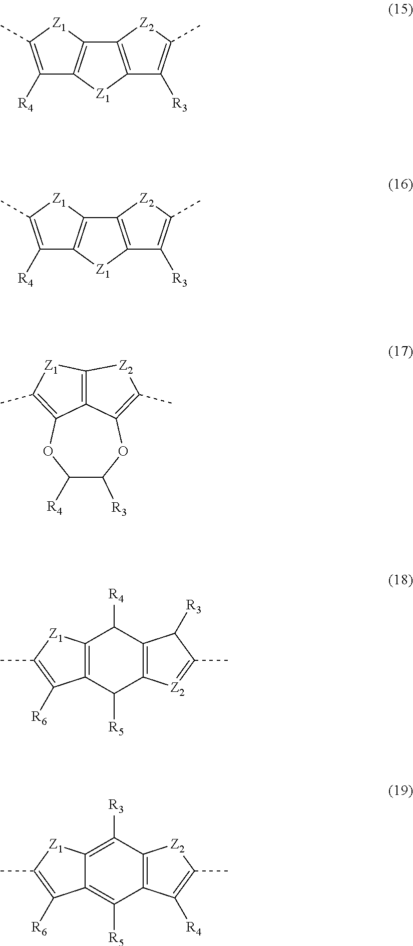
wherein
Z, Z₁, and Z₂ are independently selected from O, S, and Se, wherein Z₁ and Z₂ are different when present in the same moiety; and
R₃, R₄, R₅, and R₆ are independently selected from H, halogen, C1-C10 alkyl, C1-C10 alkoxy group, C1-C10 alkylthio (—S-alkyl), and —C≡N.

6. The dispiro-oxepine derivative according to claim 5, wherein formula (Ia) or (Ib) is selected from a moiety of formula (K1a) or (K2a):

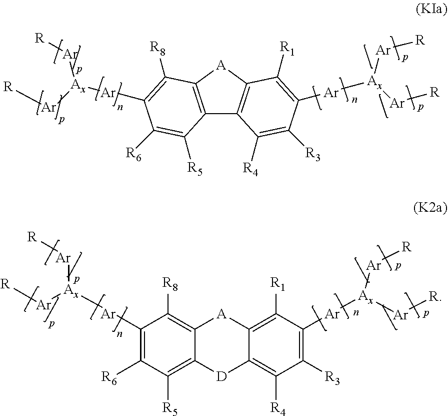

7. The dispiro-oxepine derivative according to claim 1, wherein formula (Ia) and (Ib) contains two moieties of K1, K1a, K2, K2a, or a combination thereof.

8. The dispiro-oxepine derivative according to claim 1, wherein the heteroatom is selected from O, S, and N.

9. The dispiro-oxepine derivative according to claim 8, wherein the heteroatom is selected from O and S.

10. The dispiro-oxepine derivative according to claim 7, wherein the two moieties are the same.

11. The dispiro-oxepine derivative according to claim 2, wherein any unsubstituted R₁-R₈ of formula (1) according to any one of formula (Ia), (Ib), (IIa), (IIb), (IIIa), and (IIIb) is H.

12. The dispiro-oxepine derivative according to claim 4, wherein R₉, R₁₀, R₁₁, R₁₂, R₁₃, R₁₄, R₁₅, R₁₆, R₁₇, and R₁₈ are H.

13. A hole transporting material comprising at least one compound selected from formulae (Ia), (Ib), (IIa), (IIb), (IIIa), and (IIIb) according to claim 2.

14. A dispiro-oxepine derivative for optoelectronic semiconductors, comprising a compound having the formula:

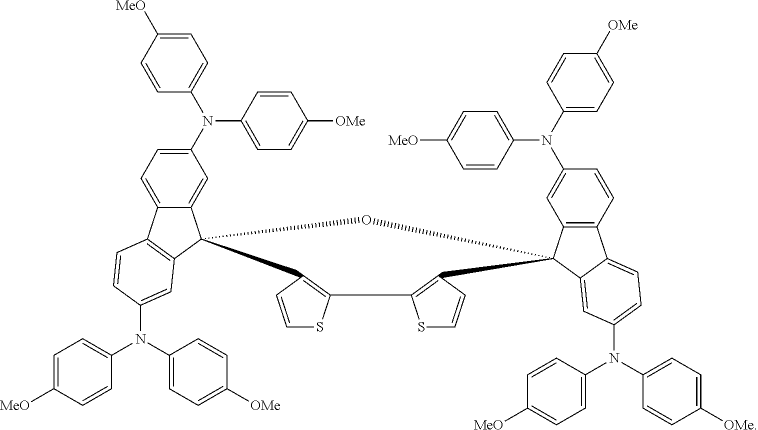

15. A semiconductor material for optoelectronic devices, comprising:
an electron transporting material (ETM) infiltrated with a perovskite absorbing material; and
a coating of the dispiro-oxepine derivative according to claim 14 disposed on the ETM.

16. The semiconductor material according to claim 15, wherein said ETM comprises a layer of mesoporous titanium dioxide (TiO₂) disposed on a thin film of TiO₂.

17. The semiconductor material according to claim 15, wherein said perovskite absorbing material comprises (FAPbI₃)_{0.85}(MAPbBr₃)_{0.15}.

18. An optoelectronic device comprising the semiconductor material according to claim 15.
